(19) United States
(12) Patent Application Publication
(10) Pub. No.: US 2005/0112193 A1
(43) Pub. Date: May 26, 2005

(54) Immediate-release formulations of acid-labile pharmaceutical compositions

(76) Inventors: Jeffrey O. Phillips, Ashland, MO (US); Ken J. Widder, Rancho Santa Fe, CA (US)

Correspondence Address: MAYER, BROWN, ROWE & MAW LLP, 190 SOUTH LASALLE ST, CHICAGO, IL, 60603-3441, US

(21) Appl. No.: 10/896,682

(22) Filed: Jul. 22, 2004

Related U.S. Application Data
(60) Provisional application No. 60/489,363, filed on Jul. 23, 2003.

(51) Int. Cl. A61K 31/4439; A61K 9/20

(52) U.S. Cl. 424/464; 514/338

(57) ABSTRACT

The present invention provides, inter alia, compositions comprising a pH buffering agent and a controlled-release component containing an acid-labile pharmaceutical agent. Methods of using such compositions are also provided.

This application claims priority of U.S. Provisional Application Ser. No. 60/489,363, filed on Jul. 23, 2003, the entirety of which is hereby incorporated by reference herein.

### FIELD OF THE INVENTION

The present invention is related to an immediate-release pharmaceutical composition containing a buffering agent and a controlled-release component containing an acid-labile pharmaceutical agent for release into gastrointestinal fluid, to methods for the manufacture of such a pharmaceutical composition, to the use of such a pharmaceutical composition in treating disease, to combinations of such a pharmaceutical composition with other therapeutic agents, and to kits containing such a pharmaceutical composition.

### BACKGROUND OF THE INVENTION

An acid-labile pharmaceutical compound must be protected from contact with acidic stomach secretions to maintain its pharmaceutical activity. The acid-labile compound upon oral administration must be transferred in intact form, that is, a non-acid degraded or reacted form, to the location in the gastrointestinal tract where the pH is near or above its pKa and where absorption of the acid-labile pharmaceutical compound can occur. Typically, these acid-labile compounds are formulated for oral delivery into the intestine as gastrointestinal fluid resistant enteric coated solid dosage form, or as a delayed- or controlled-release capsule or tablet, or as an intravenous solution (or as a product for reconstitution).

A class of acid-labile pharmaceutical compounds that are administered as enteric coated dosage forms are proton pump inhibiting agents. Examples include, omeprazole (Prilosec®), lansoprazole (Prevacid®), esomeprazole (Nexium®, rabeprazole (Aciphex®), pantoprazole (Protonix>), pariprazole, tenatoprazole, and leminoprazole. Omeprazole is a substituted benzimidazole, 5-methoxy-2-[ (4-methoxy-3,5-dimethyl-2-pyridinyl) methyl] sulfinyl]-1H-benzimidazole, that inhibits gastric acid secretion. Omeprazole belongs to a class of antisecretory compounds that do not exhibit anti-cholinergic or H2 histamine antagonist properties. Drugs of this class suppress gastric acid secretion by the specific inhibition of the H⁺, K⁺-ATPase enzyme system (proton pump) at the secretory surface of the gastric parietal cell.

U.S. Pat. No. 4,786,505 to Lovgren et al. teach that a pharmaceutical oral solid dosage form of omeprazole must be protected from contact with acidic gastric juice by an enteric coating to maintain its pharmaceutical activity. This patent describes an enteric coated omeprazole preparation containing a separating subcoat between the core material and the enteric coating. The preparation contains an alkaline core comprising omeprazole, a subcoating and an enteric coating.

Typically, omeprazole, lansoprazole and other acid-labile proton pump inhibitors are formulated in an enteric coated solid dosage form (as either a delayed-release granule in a capsule, tablet or packet) or as an intravenous solution (as a product for reconstitution), and are prescribed for short-term treatment of active duodenal ulcers, gastric ulcers, gastroesophageal reflux disease (GERD), severe erosive esophagitis, poorly responsive symptomatic GERD, and pathological hypersecretory conditions such as Zollinger Ellison syndrome. These conditions are caused by an imbalance between acid and pepsin production, called aggressive factors, and mucous, bicarbonate, and prostaglandin production, called defensive factors. These above-listed conditions commonly arise in healthy or critically ill patients, and may be accompanied by significant upper gastrointestinal bleeding.

Lansoprazole is available for oral administration as granules in a gelatin capsule or a single use packet. Other proton pump inhibiting agents such as rabeprazole and pantoprazole are supplied as enteric coated tablets. The enteric dosage forms have been employed because it is important that these proton pump inhibitors not be exposed to low pH gastric acid prior to absorption. Although these proton pump inhibitors are stable at alkaline pH, they are degraded rapidly as pH falls. Therefore, if the microencapsulation or the enteric coating is disrupted (for example, trituration to compound a liquid, or chewing the capsule or tablet), the drug will be exposed to degradation by the gastric acid in the stomach.

It is believed that omeprazole, lansoprazole and other proton pump inhibiting agents reduce gastric acid production by inhibiting H⁺, K⁺-ATPase of the parietal cell—the final common pathway for gastric acid secretion (Fellenius et al., Substituted Benzimidazoles Inhibit Gastric Acid Secretion by Blocking H⁺, K⁺-ATPase, NATURE, 290:159-161(1981); Wallmark et al, The Relationship Between Gastric Acid Secretion and Gastric H⁺, K⁺-ATPase Activity, J. BIOL. CHEM., 260: 13681-13684 (1985); Fryklund et al., Function and Structure of Parietal Cells After H⁺, K⁻ATPase Blockade, AM. J. PHYSIOL., 254 (3 PT 1); G399-407 (1988)). The substituted benzimidazole proton pump inhibiting agents contain a sulfinyl group in a bridge between substituted benzimidazole and pyridine rings.

At neutral pH, these proton pump inhibiting agents are chemically stable, lipid-soluble compounds that have little or no inhibitory activity. These neutral compounds reach parietal cells from the blood and diffuse into the secretory canaliculi, where the drugs become protonated and thereby trapped. The protonated agent rearranges to form a sulfenic acid and a sulfenamide. The sulfenamide interacts covalently with sulfhydryl groups at critical sites in the extracellular (luminal) domain of the membrane-spanning H⁺, K⁺-ATPase. See, Hardman et al., Goodman & Gilman's The Pharmacological Basis of Therapeutics, p. 907, 9^{th} ed. (1996). These proton pump inhibiting agents, therefore, are considered prodrugs that must be activated to be effective. The specificity of the effects of proton pump inhibiting agents is also dependent upon: (a) the selective distribution of H⁺, K⁺-ATPase; (b) the requirement for acidic conditions to catalyze generation of the reactive inhibitor; and (c) the trapping of the protonated drug and the cationic sulfenamide within the acidic canaliculi and adjacent to the target enzyme. See, for example, Hardman et al., (1996).

The term “enteric coating” as used in the art prior to the current invention refers to a gastric acid resistant, enterosoluble coating for drug release in the intestine. Enteric coatings generally relate to a mixture of pharmaceutically acceptable excipients which are applied to, combined with, mixed with or otherwise added to a carrier or composition. The coating may be applied to, for example, a compressed or molded or extruded tablet, a gelatin capsule, and/or pellets, beads, granules or particles of the carrier or composition. The coating may be applied through an aqueous dispersion or after dissolving in appropriate solvent.

Various enteric materials, for example, cellulose acetate phtbalate, hydroxypropyl methylcellulose phthalate, polyvinyl acetate phthalate, and various Eudragit™ acrylic polymers, have been used as enteric coatings. The enteric materials, which are soluble at higher pH values, are frequently used for colon-specific dosage forms. I

Enteric coatings and the selection of their properties depend on several considerations including the ability to dissolve or disintegrate rapidly at the target intestine site. In order for this to occur the enteric coating must be resistance to dissolution and disintegration in the stomach, and be impermeable to gastric fluids while in the stomach. These properties also contribute to certain physical and chemical stability characteristics during manufacturing and storage. Typical dosage forms of enteric coated compositions are formulated as enteric coated delayed release oral dosage forms, that is, as an oral dosage form of a pharmaceutical composition which utilizes an enteric coating to effect release in the lower gastrointestinal tract.

However, due to their pH-dependent attributes and the uncertainty of gastric retention time, in-vivo performance as well as inter- and intra-subject variability are major issues for using enteric coated systems for controlled release of a drug.

Therefore, there is a need for additional controlled-release formulations that release an acid-labile pharmaceutical agent into the gastrointestinal tract for immediate absorption of an intact, non-acid degraded or non-acid reacted form into the bloodstream of a subject. A faster onset of action, a more timely and consistent absorption profile, improved side-effect profile, reduced dosing amount and frequency, and improved patient compliance are also desired for the controlled release dosage forms that release acid-labile drug into the stomach for absorption, as compared to delay-release preparations that release drug in the intestine for absorption. The discussion that follows discloses pharmaceutical compositions containing acid-labile compounds that help to fulfill these needs.

### SUMMARY OF THE INVENTION

The effective oral administration to a subject of an acid labile pharmaceutical agent, such as a proton pump inhibiting agent, has been complicated by the compound's acid lability in gastrointestinal fluid, as well as by its other physical and chemical properties. Pharmaceutical compositions comprising a buffering agent and a controlled-release component, however, have been discovered that can effectively deliver a therapeutically-effective amount of the pharmaceutical agent to the subject. In one embodiment, a composition containing a pH buffering agent and a controlled-release component containing an acid-labile pharmaceutical agent affect release of the acid labile pharmaceutical agent into gastrointestinal fluid upon exposure of the composition to the gastrointestinal fluid, such as, for example, after orally administering the composition to a subject or testing the composition in an in vitro stomach model. These combinations of a buffering agent and a controlled-release component possess improved bioavailability, and/or pharmacokinetic, pharmacodynamic, chemical or physical properties. The present invention comprises these pharmaceutical compositions, dosage forms and kits based thereon, and methods for the preparation and use thereof.

### DETAILED DESCRIPTION OF THE INVENTION

The present invention is directed to methods, kits, combinations, and compositions for treating a condition or disorder where treatment with an acid-labile pharmaceutical agent is indicated. In one embodiment of the present invention, the acid-labile pharmaceutical agent is an H⁺, K⁺-ATPase inhibiting agent or inhibitor, such as, for example, a proton pump inhibiting agent.

In therapy of a condition or disorder, it is important to provide a dosage form that delivers the required therapeutic amount of the drug in vivo, and renders the drug bioavailable in a rapid manner. The formulations of the present invention satisfy these needs.

While the present invention may be embodied in many different forms, several specific embodiments are discussed herein with the understanding that the present disclosure is to be considered only as an exemplification of the principles of the invention, and it is not intended to limit the invention to the embodiments illustrated. Where the invention is illustrated herein with particular reference to proton pump inhibiting agent or an H⁺, K⁺-ATPase inhibiting agent, it will be understood that any other acid-labile pharmaceutical agent can, if desired, be substituted in whole or in part for the proton pump inhibiting agent or H⁺, K⁺-ATPase inhibiting agent in the methods, kits, combinations, and compositions herein described. Where the invention is illustrated herein with particular reference to sodium bicarbonate, and/or sodium carbonate, and/or calcium carbonate as a buffering agent, it will be understood that any other buffering agent can, if desired, be substituted in whole or in part for sodium bicarbonate, and/or sodium carbonate, and/or calcium carbonate in the methods, kits, combinations, and compositions herein described.

It has been discovered that pharmaceutical compositions comprising a pH buffering agent and a controlled-release component containing an acid-labile pharmaceutical agent, for example, an , K⁺-ATPase inhibiting agent, along with an optional pharmaceutically acceptable carrier material are unique compositions exhibiting improved performance as acid-labile pharmaceutical agents. Such pharmaceutical compositions exhibit improved onset of action, a more timely and consistent absorption profile, improved side effect profile, reduced dosing and amount of frequency, and improved patient compliance. In one embodiment of the present invention, these compositions provide an H⁺, K⁺-ATPase inhibiting agent or inhibitor to a subject in a dosage that is sufficient to provide prolonged inhibition of H⁺, K⁺-ATPase and thus confer the desired therapeutic benefit. Undesirable side effects such as, but not limited to, gastrointestinal irritation, are also minimized with the pharmaceutical compositions of the present invention.

In one embodiment, the composition contains a pH buffering agent (“buffering agent”) and a controlled-release component that contains an acid-labile pharmaceutical agent, each for release into gastrointestinal fluid, which includes, for example, the fluid content of a subject's stomach, which may include, for example, saliva or an aqueous medium containing bile salts and enzymes. Illustratively, the buffering agent is in an amount effective at buffering gastrointestinal fluid to a predetermined pH for a predetermined length of time, and the controlled-release component contains a gastrointestinal-disorder-effective amount of an acid-labile pharmaceutical agent.

In one embodiment, the condition or disorder is a gastrointestinal disorder. Illustratively, the gastrointestinal disorder is an acid-caused gastrointestinal disorder, and includes, for example, a duodenal ulcer disease, a gastric ulcer disease, a gastroesophageal reflux disease, an erosive esophagitis, a poorly responsive symptomatic gastroesophageal reflux disease, a pathological gastrointestinal hypersecretory disease, Zollinger Ellison Syndrome, acid dyspepsia, heartburn, an esophageal disorder, a non-erosive reflux disorder, and/or a NSAID induced ulcer. In one embodiment of the present invention, the gastrointestinal disorder is heartburn. Illustratively, the heartburn can be meal related or induced, and/or sleep related or induced, and/or nighttime related or induced. Sleep related or induced heartburn and/or nighttime related or induced heartburn can be caused, for example, by breakthrough gastritis between conventional doses of a therapeutic agent, such as while sleeping or in the early morning hours after a night's sleep. Treatment of these conditions is accomplished by administering to a subject a gastrointestinal-disorder-effective amount (or a therapeutically-effective amount) of a pharmaceutical composition according to the present invention. The subject may be experiencing one or more of these conditions or disorders.

The present invention is also directed to methods, kits, combinations, and compositions for treating, preventing, or reducing the risk of developing a gastrointestinal disorder, or the symptoms associated with, or related to a gastrointestinal disorder in a subject in need thereof.

Also included in the methods, kits, combinations, and compositions of the present invention is a pharmaceutical composition comprising a controlled-release component containing a gastrointestinal-disorder-effective amount of an acid-labile pharmaceutical agent and a controlled-release layer comprising an enteric coating that covers, coats, or layers the acid-labile pharmaceutical agent. In one embodiment of the present invention, the composition further comprises at least one uncoated acid-labile pharmaceutical agent. In another embodiment of the present invention the controlled-release component comprises an enteric coated proton pump inhibiting agent. In still another embodiment, the composition comprises a proton pump inhibiting agent optionally coated with an enteric coating and a buffering agent. See, for example, U.S. Pat. No. 6,489,346 for examples of non-enteric dosage forms. The term “enteric coating” as generally defined, for example in “Remington: The Science and Practice of Pharmacy”, refers to coatings that remain intact in the stomach but dissolve and release the contents of the dosage form once it reaches the small intestine. As used herein, the term “enteric coating” includes sustained- or controlled-release coatings (e.g. hydroxypropylmethylcellulose) and coatings that are made of traditional enteric coating materials (e.g. hydroxypropylmethylcellulose phthalate) but which, because of thickness or other physical (e.g. mechanical properties, uniformity, etc.) and/or chemical properties, dissolves or disintegrates in the upper gastrointestinal tract and/or stomach. Illustratively, such a coating may be provided that protects the PPI from degradation under ambient conditions, for example during packaging, shipping and storage of finished dosage forms, while still allowing for dissolution or disintegration of the coating in the gastrointestinal fluids as described elsewhere herein. Additionally, such a coating may also provide taste masking attributes.

In one embodiment of the present invention, the composition is formulated to provide a buffering agent and a controlled-release component that contains an acid-labile pharmaceutical agent for release into gastrointestinal fluid, for example, upon oral administration of the composition to a subject, or upon exposure to gastrointestinal fluid upon testing in an in vitro stomach model. In yet another embodiment, the one or more buffering agents elevate pH of gastrointestinal fluid to a pH where the controlled-release layer substantially dissolves, which causes the acid-labile pharmaceutical agent to be released into the gastrointestinal fluid where, for example, absorption into the bloodstream occurs.

In one embodiment, the pharmaceutical composition comprises a pH-dependent film or coating that retards or delays the release of the acid-labile pharmaceutical agent into gastrointestinal fluid until a predetermined pH is reached (“delayed release” or “delay time”). Once this predetermined pH is reached, the release of the pharmaceutical agent from the controlled-release component into the gastrointestinal fluid is rapid and, in an in vivo system, is available for absorption into the blood serum.

In one embodiment, once the predetermined pH is reached in either an in vivo or in vitro system, substantially the entire amount of pharmaceutical agent is released from the composition into gastrointestinal fluid within less than about 90 minutes, or within less than about 60 minutes, or within less than about 50 minutes, or within less than about 40 minutes, or within less than about 30 minutes, or within less than about 20 minutes, or within less than about 15 minutes, or within less than about 10 minutes, or within less than about 5 minutes under a predetermined environmental pH condition. In another embodiment of the present invention, the enteric coating is pH dependent, which rapidly disintegrates in gastrointestinal fluid having a predetermined pH.

In various embodiments of the present invention, the predetermined pH is between about 3 to about 6, or between about 3 to about 8, or between about 4 to about 8, or between about 4 to about 7, or between about 5 to about 8, or between 5 to about 7, or greater than about 3, or about 3.5, or about 4, or about 4.5, or about 5, or about 5.5, or about 6, or about 6.5, or about 7, or about 7.5, or about 8.

In other embodiments, the pH of the gastrointestinal fluid in either an in vivo or in vitro system is maintained for a time period after exposure of the composition to the gastrointestinal fluid that substantially dissolves the pH-dependent film or coating. In further embodiments of the present invention, once the predetermined pH is reached, the release of the pharmaceutical agent from the controlled-release component into the gastrointestinal fluid is rapid and substantially complete releasing about 1% to about 85% of the acid-labile pharmaceutical agent from the composition, or not less than about 85%, or not less than about 80%, or not less than about 75%, or not less than about 70%, or not less than about 65%, or not less than about 60%, or not less than about 55%, or not less than about 50%, or not less than about 45%, or not less than about 40% of the acid-labile pharmaceutical agent into the gastrointestinal fluid within about 90 minutes.

The delay time takes into consideration factors such as transit times, type, thickness and composition of the enteric coating, the use of various types and combinations of buffering agents, and/or food effects, general health, age, sex and diet of the subject, the time of day the composition administered, or the use of antacids or other medicaments, which alter the pH of the gastrointestinal tract.

The pharmaceutical agent of the present invention is administered and dosed in accordance with good medical practice, taking into account the clinical condition of the individual patient, the site and method of administration, scheduling of administration, and other factors known to medical practitioners.

In one aspect, the present invention is directed to therapeutic methods of treating a condition, disease or disorder where treatment with an H⁺, K⁺-ATPase inhibiting agent or inhibitor is indicated, the method comprises oral administration of one or more compositions of the present invention to a subject in need thereof in an amount effective at treating the condition, disease, or disorder. In one embodiment, the condition or disorder is a gastrointestinal disorder. The dosage regimen to prevent, give relief from, or ameliorate the condition or disorder can be modified in accordance with a variety of factors. These factors include the type, age, weight, sex, diet, and medical condition of the subject and the severity of the disorder or disease. Thus, the dosage regimen actually employed can vary widely and therefore can deviate from the dosage regimens set forth herein.

The present invention also includes methods of treating, preventing, reversing, halting or slowing the progression of a gastrointestinal disorder once it becomes clinically evident, or treating the symptoms associated with, or related to the gastrointestinal disorder, by administering to the subject a composition of the present invention. The subject may already have a gastrointestinal disorder at the time of administration, or be at risk of developing a gastrointestinal disorder. The symptoms or conditions of a gastrointestinal disorder in a subject can be determined by one skilled in the art and are described in standard textbooks. The method comprises the oral administration a gastrointestinal-disorder-effective amount of one or more compositions of the present invention to a subject in need thereof.

The term “gastrointestinal-disorder-effective amount” means the amount of pharmaceutical agent effective to achieve a pharmacologic effect or therapeutic improvement without undue adverse side effects, including but not limited to, raising gastrointestinal fluid pH, reducing gastrointestinal bleeding, reducing the need for blood transfusion, improving survival rate, more rapid recovery, parietal cell activation and H⁺, K⁺-ATPase inhibition, or improving or eliminating symptoms and other indicators as are selected as appropriate measures by those skilled in the art, without undue adverse side effects.

The term “treat” or “treatment” as used herein refers to any treatment of a disorder or disease associated with a gastrointestinal disorder, and includes, but is not limited to, preventing the disorder or disease from occurring in a subject which may be predisposed to the disorder or disease, but has not yet been diagnosed as having the disorder or disease; inhibiting the disorder or disease, for example, arresting the development of the disorder or disease; relieving the disorder or disease, for example, causing regression of the disorder or disease; or relieving the condition caused by the disease or disorder, for example, stopping the symptoms of the disease or disorder.

The term “prevent” or “prevention,” in relation to a gastrointestinal disorder or disease, means no gastrointestinal disorder or disease development if none had occurred, or no further gastrointestinal disorder or disease development if there had already been development of the gastrointestinal disorder or disease.

Besides being useful for human treatment, the present invention is also useful for other subjects including veterinary animals, reptiles, birds, exotic animals and farm animals, including mammals, rodents, and the like. Mammals include primates, for example, a monkey, or a lemur, horses, dogs, pigs, or cats. Rodents include rats, mice, squirrels, or guinea pigs.

Also included in the methods, kits, combinations, and compositions of the present invention is a pharmaceutical composition comprising a buffering agent in an amount sufficient to increase pH of gastrointestinal fluid to a pH that substantially prevents or inhibits acid degradation of the acid-labile pharmaceutical agent, for example, a proton pump inhibiting agent, for a period of time.

In one embodiment, the time period that the pH is elevated facilitates absorption of a therapeutically-effective amount of a substantially non-acid degraded or non-acid reacted acid-labile pharmaceutical agent from the stomach into the blood serum of a subject. Illustratively, the amount of intact drug absorbed into the serum is greater than the absorption of intact drug in the absence of the buffering agent when administered to a subject. In another embodiment of the present invention, the amount of intact drug that is absorbed into the bloodstream is greater than about 50 percent, or greater than about 75 percent, or greater than about 80 percent, or greater than about 85 percent, or greater than about 90 percent, or greater than about 95 percent, or greater than about 99 percent of the total amount of acid-labile pharmaceutical agent present in the composition and administered to a subject.

In yet another embodiment, in an in vivo and/or an in vitro model, a composition of the present invention substantially maintains these respective percentages of intact, non-acid degraded or non-acid reacted acid labile pharmaceutical agent in gastrointestinal fluid for a time period of about 90 minutes or less, or less than about 45 minutes, or less than about 30 minutes, or less than about 20 minutes, or less than about 15 minutes, or less than about 10 minutes, or about 10 minutes, or about 11 minutes, or about 12 minutes, or about 13 minutes, or about 14 minutes, or about l 5 minutes, or about 16 minutes, or about 17 minutes, or about 18 minutes, or about 19 minutes, or about 20 minutes, or about 22.5 minutes, or about 25 minutes, or about 27.5 minutes, or about 30 minutes, or about 32.5 minutes, or about 35 minutes, or about 37.5 minutes, or about 40 minutes, or about 42.5 minutes, or about 45 minutes, or about 47.4 minutes, or about 50 minutes, or about 60 minutes, or about 70 minutes, or about 80 minutes, or about 90 minutes.

After oral administration to a subject, a portion of intact drug is absorbed into the bloodstream during this time period. Illustratively, the Tₘₐₓ of an acid labile pharmaceutical agent, for example, a proton pump inhibiting agent, is reached within about 30 seconds to about 90 minutes, or within about 10 minutes, or within about 15 minutes, or within about 20 minutes, or within about 25 minutes, or within about 30 minutes, or within about 35 minutes, or within about 40 minutes, or within about 45 minutes, or within about 50 minutes, or within about 55 minutes, or within about 60 minutes, or within about 65 minutes, or within about 70 minutes, or within about 75 minutes, or within about 80 minutes, or within about 85 minutes, or within about 90 minutes after administration to a subject.

Generally speaking, the illustrated percentages of intact drug that is absorbed into the bloodstream is not narrowly critical, as long as a therapeutic-disorder-effective amount, for example, a gastrointestinal-disorder-effective amount of a proton pump inhibiting agent, is absorbed following administration of the composition to a subject.

In one embodiment of the present invention, the composition is administered in an amount to achieve a measurable serum concentration of a non-acid degraded proton pump inhibiting agent greater than about 0.1 μg/ml within about 45 minutes, or within about 30 minutes, or within about 25 minutes, or within about 20 minutes, or within about 17.5 minutes, or within about 15 minutes, or within about 12.5 minutes, or within about 10 minutes, or within about 7.5 minutes, or within about 5 minutes after administration of the composition.

In another embodiment of the present invention, the composition is administered to the subject in an amount to maintain a measurable serum concentration of a non-acid degraded or non-acid reacted proton pump inhibiting agent greater than about 0.1 μg/ml from at least about 15 minutes to about 3 hours after administration of the composition. In another embodiment of the present invention, the composition is administered to the subject in an amount to maintain a measurable serum concentration of a non-acid degraded or non-acid reacted proton pump inhibiting agent greater than about 0.1 μg/ml from at least about 15 minutes to at least about 90 minutes after administration of the composition, or from at least about 15 minutes to about 1 hour after administration of the composition; or from at least about 15 minutes to about 45 minutes after administration of the composition; or from at least about 15 minutes to about 30 minutes after administration of the composition.

In another embodiment of the present invention, the composition is administered to the subject in an amount to achieve an initial serum concentration of a non-acid degraded or non-acid reacted proton pump inhibiting agent greater than about 0.1 μg/ml from at least about 30 minutes to about 1 hour after administration of the composition; or greater than about 0.1 μg/ml from at least about 30 minutes to about 45 minutes after administration of the composition.

In still another embodiment of the present invention, the composition is administered to the subject in an amount to achieve a measurable serum concentration of a non-acid degraded or non-acid reacted proton pump inhibiting agent greater than about 0.1 μg/ml, or about 1.5 μg/ml, or about 0.2 μg/ml, or about 0.3 μg/ml, or about 0.4 μg/ml, or about 0.5 μg/ml, or about 0.6 μg/ml, or about 0.7 μg/ml, or about 0.8 μg/ml, or about 0.9 μg/ml, or about 1 μg/ml within about 1.5 hours, or within about 75 minutes, or within about 60 minutes, or within about 55 minutes, or within about 50 minutes, or within about 45 minutes, or within about 40 minutes, or within about 35 minutes, or within about 30 minutes, or within about 25 minutes, or within about 20 minutes, or within about 17 minutes, or within about 15 minutes, or within about 12 minutes, or within about 10 minutes after administration of the composition to a subject.

In other embodiments of the present invention, the composition upon ingestion by a subject provides an initial blood serum concentration of about 0.15 μg proton pump inhibiting agent/ml in a subject within about 15 minutes after ingestion In another embodiment, the composition upon ingestion by a subject provides an initial blood serum concentration of about 0.15 μg proton pump inhibiting agent/ml within about 20 minutes after ingestion. In yet another embodiment of the present invention, the composition upon ingestion by a subject provides an initial blood serum concentration of about 0.15 μg proton pump inhibiting agent/ml within about 30 minutes after ingestion. In yet another embodiment of the present invention, the composition upon ingestion by a subject provides an initial blood serum concentration of about 0.15 μg proton pump inhibiting agent/ml is achieved within about 45 minutes after ingestion.

In yet another embodiment of the present invention, the composition upon ingestion by a subject provides a therapeutic effect as a proton pump inhibiting agent in a subject over an interval of about 30 minutes to about 24 hours after ingestion.

The phrase “gastrointestinal fluid” refers to the fluid of stomach secretions of a subject or the saliva of a subject after oral administration of a composition of the present invention, or the equivalent thereof. An equivalent of stomach secretion includes, for example, an in vitro fluid having similar content and/or pH as stomach secretions including, for example, a 1% sodium dodecyl sulfate solution for less acid, neutral, or basic test solutions; 0.1N hydrochloric acid in water for more acidic test solutions; or simulated gastrointestinal fluid, USP 26-NF 21. The content and pH of a particular stomach secretion is generally subject specific, and depends upon, among other things, the weight, sex, age, diet, or health of a particular subject. These equivalents of stomach secretions can, for example, be mimicked or replicated by those skilled in the art. One such model, described more fully below, is commonly known as the “Kinetic Acid Neutralization Model,” and can be used to experimentally study or determine release kinetics (for example, immediate-release versus control release kinetics) of a component of the compositions of the present invention under predetermined experimental conditions; or acid degradation of an acid-labile pharmaceutical agent of the compositions herein described under predetermined experimental conditions. See, for example, Beekman S M, Preparation and Properties of New Gastric Antacids, I. J. Pharm Assoc 1960; 49; 191-200; Fuchs C., Antacids, Their Function, Formulation and Evaluation, Drug Cosmetic Ind. 1949; 64;692-773. The in vitro stomach model can also be use to simulate fed or unfed conditions of a subject, for example, by including oils or fatty substances to simulate a fed state.

In another embodiment of the present invention, the composition of the present invention comes in the form of a kit or package containing one or more of the compositions or therapeutic agents of the present invention. The composition containing the composition or therapeutic agent can be packaged in the form of a kit or package in which hourly, daily, weekly, or monthly (or other periodic) dosages are arranged for proper sequential or simultaneous administration. The present invention further provides a kit or package containing a plurality of dosage units, adapted for successive daily administration, each dosage unit comprising at least one of the compositions or therapeutic agents of the present invention. This drug delivery system can be used to facilitate administration of any of the various embodiments of the compositions and therapeutic agents of the present invention. In one embodiment, the system contains a plurality of doses to be to be administered daily or as needed for symptomatic relief. The kit or package can also contain agents utilized in combination therapy to facilitate proper administration of the dosage forms. The kit or package can also contain a set of instructions for the subject.

As used herein, the phrase “acid-labile pharmaceutical agent” refers to any pharmacologically active drug subject to acid catalyzed degradation. The term “pharmacologically active drug” and its equivalents, includes at least one of any therapeutically, prophylactically and/or pharmacologically or physiologically beneficial active substance, or mixture thereof, which is delivered to a living subject to produce a desired, usually therapeutic, effect. More specifically, any drug which is capable of producing a pharmacological response, localized or systemic, irrespective of whether therapeutic, diagnostic, or prophylactic in nature, particularly in mammal, is within the contemplation of the invention. It should be noted that the drugs and/or bioactive agents may be used singularly or as a mixture of two or more such agents, and in amounts sufficient to prevent, cure, diagnose or treat a disease or other condition, as the case may be.

While not wishing to be bound by theory, the acidic decomposition of an acid-labile compound is believed to be due to an acid catalyzed reaction as described, for example, by G. Rackur et al., in Biochem. Biophys. Res. Commun. 1985: 128(1). P477-484. Thus, the pharmacologically active agents useful in the present invention are those which are degraded by acids, even organic acids, or are degraded in acid catalyzed reactions. Examples of acid-labile pharmacologically active agents useful in the present invention, include, for example, the compounds disclosed in EP 244 380; U.S. Pat. No. 4,045,563; EP-0 005 129; EP-0 166 287; EP-0 174 726; EP-0 184 322; RP-0 261 478; EP-0 268 956; BE-898 880; GB-2 141 429; EP-0 146 370; GB-2 082 580; EP-A-0 173 664; EP-A-0 080 602; EP-0127 763; EP-0 134 400; EP-0 130 729; EP-0 150 586; DE-34 15971; GB-2 082 580; SE-A-8504048-3 and U.S. Pat. No. 4,182,766; substituted phenylmethylsulfinyl-1H-benzimidazoles, cycloheptapyridin-9-yisulfinyl-1H-benzimidazoles or pyridin-2-yimethylsulfinylthienoimidazoles, as disclosed in DE-A-35 31 487, EP-A-0 434 999 or EP-A-0 234 485; and 2-[2-(N-isobutyl-N-methylamino)benzylsulfinyl] benzimidazole (leminoprazole) and 2-(4-methoxy-6,7,8,9-tetrahyd-ro-5H-cycloheptafblpyridin-9-ylsulfinyl)-1H-benzimidazole (nepaprazole).

A class of acid-labile pharmaceutical agents useful in the methods, kits, combinations, and compositions of the present invention include an agent possessing pharmacological activity as an inhibitor of H⁺, K⁺-ATPase. The term “proton pump inhibitor,” or “PPI,” or “proton pump inhibiting agent” means any agent possessing pharmacological activity as an inhibitor of H⁺, K⁺-ATPase. The definition of “PPI,” or “proton pump inhibitor,” or “proton pump inhibiting agent” as used herein can also mean that the agent possessing pharmacological activity as an inhibitor of H⁺,K⁺-ATPase may, if desired, be in the form of a free base, a free acid, a salt, an ester, a hydrate, an amide, an enantiomer, an isomer, a tautomer, a prodrug, a polymorph, a derivative or the like, provided the free base, free acid, salt, ester, hydrate, amide, enantiomer, isomer, tautomer, prodrug, polymorph, or derivative is suitable pharmacologically, that is, effective in the present methods, combinations, kits, and compositions.

A class of proton pump inhibiting agents useful in the methods, kits, combinations, and compositions of the present invention includes substituted benzimidazole compounds possessing such pharmacological activity as an inhibitor of H⁺, K⁺-ATPase, including a substituted benzimidazole, for example, a proton pump inhibiting agent.

In yet another embodiment, the inhibitor of H⁺, K⁺-ATPase is of the formula (I):

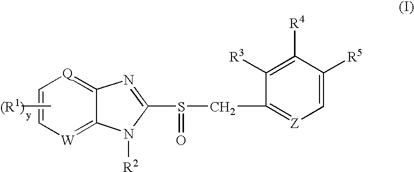

R¹ is hydrogen, alkyl, halogen, cyano, carboxy, carboalkoxy, carboalkoxyalkyl, carbamoyl, carbamoylalkyl, hydroxy, alkoxy, hydroxyalkyl, trifluoromethyl, acyl, carbamoyloxy, nitro, acyloxy, aryl, aryloxy, alkylthio, or alkylsulfinyl;

R² is hydrogen, alkyl, acyl, acyloxy, alkoxy, amino, aralkyl, carboalkoxy, carbamoyl, alkylcarbamoyl, dialkylcarbamoyl, alkylcarbonylmethyl, alkoxycarbonylmethyl, or alkylsulfonyl;

R³ and R⁵ are the same or different and each is hydrogen, alkyl, alkoxy, amino, or alkoxyalkoxy;

R⁴ is hydrogen, alkyl, alkoxy which may optionally be fluorinated, or alkoxyalkoxy;

Q is nitrogen, CH, or CR¹;

W is nitrogen, CH, or CR¹;

y is an integer of 0 through 4; and

Z is nitrogen, CH, or CR¹;

or a salt, an ester, a hydrate, an amide, an enantiomer, an isomer, a tautomer, a polymorph, a polymorph, a prodrug, or a derivative thereof.

Illustratively, a compound of interest that can be used in the methods, kits, combinations, and compositions of the present invention includes, but is not limited to, omeprazole, tenatoprazole, lansoprazole, rabeprazole, esomeprazole, pantoprazole, pariprazole, and leminoprazole; as a free base, a free acid, or a salt, hydrate, ester, amide, enantiomer, isomer, tautomer, polymorph, prodrug, or derivative of these compounds. (Based in part upon the list provided in The Merck Index, Merck & Co. Rahway, N.J. (2001)).

Other compounds of interest include, for example, soraprazan (Altana); ilaprazole (U.S. Pat. No. 5,703,097) (II-Yang); AZD-0865 (AstraZeneca); YH-1885 (PCT Publication WO 96/05177) (SB-641257) (2-pyrimidinamine, 4-(3,4-dihydro-1 -methyl-2(1H)-isoquinolinyl)-N-(4-fluorophenyl)-5,6-dimethyl-, monohydrochloride) (YuHan); BY-112 (Altana); SPI-447 (Imidazo(1,2-a)thieno(3,2-c)pyridin-3-amine,5-methyl-2-(2-methyl-3-thienyl) (Shinnippon); 3-hydroxymethyl-2-methyl-9-phenyl-7H-8,9-dihydro-pyrano(2,3-c)-imidazo(1,2-a)pyridine (PCT Publication WO 95/27714) (AstraZeneca); Pharmaprojects No. 4950 (3-hydroxymethyl-2-methyl-9-phenyl-7H-8,9-dihydro-pyrano(2,3-c)-imidazo(1,2-a)pyridine) (AstraZeneca, ceased) WO 95/27714; Pharmaprojects No. 4891 (EP 700899) (Aventis); Pharmaprojects No. 4697 (PCT Publication WO 95/32959) (AstraZeneca); H-335/25 (AstraZeneca); T-330 (Saitama 335) (Pharmacological Research Lab); Pharmaprojects No. 3177 (Roche); BY-574 (Altana); Pharmaprojects No. 2870 (Pfizer); AU-1421 (EP 264883) (Merck); AU-2064 (Merck); AY-28200 (Wyeth); Pharmaprojects No. 2126 (Aventis); WY-26769 (Wyeth); pumaprazole (PCT Publication WO 96/05199) (Altana); YH-1238 (YuHan); Pharmaprojects No. 5648 (PCT Publication WO 97/32854) (Dainippon); BY-686 (Altana); YM-020 (Yamanouchi); GYKI-34655 (Ivax); FPL-65372 (Aventis); Pharmaprojects No. 3264 (EP 509974) (AstraZeneca); nepaprazole (Toa Eiyo); HN-11203 (Nycomed Pharma); OPC-22575; pumilacidin A (BMS); saviprazole (EP 234485) (Aventis); SKandF-95601 (GSK, discontinued); Pharmaprojects No. 2522 (EP 204215) (Pfizer); S-3337 (Aventis); RS-13232A (Roche); AU-1363 (Merck); SKandF-96067 (EP 259174) (Altana); SUN 8176 (Daiichi Pharma); Ro-18-5362 (Roche); ufiprazole (EP 74341) (AstraZeneca); and Bay-p-1455 (Bayer); as a free base, a free acid, or a salt, hydrate, ester, amide, enantiomer, isomer, tautomer, polymorph, prodrug, or derivative of these compounds.

Still other proton pump inhibiting agents of interest for inhibiting gastric acid secretion are described in the patents listed in Table No. 1.

TABLE NO. 1
Issued U.S. Patents Teaching
Proton Pump Inhibiting Agents

| U.S. | | | |
|---|---|---|---|
| Pat. No. | Inventor | Filing Date | Issue Date |
| 4,628,098 | Nohara, et al. | Jul. 29 1985 | Dec. 9, 1986 |
| 4,689,333 | Nohara, et al. | Dec. 2, 1986 | Aug. 25, 1987 |
| 4,786,505 | Lovgren, et al. | Apr. 20, 1987 | Nov. 22, 1988 |
| 4,853,230 | Lovgren, et al. | Apr. 20, 1987 | Aug. 1, 1989 |
| 4,965,269 | Brandstrom, et al. | Dec. 20, 1989 | Oct. 23, 1990 |
| 5,021,433 | Alminger, et al. | May 12, 1988 | Jun. 4, 1991 |
| 5,026,560 | Makino, et al. | Jan. 14, 1988 | Jun. 25, 1991 |
| 5,045,321 | Makino, et al. | Feb. 13, 1997 | Sep. 3, 1991 |
| 5,093,132 | Makino, et al. | Aug. 31, 1990 | Mar. 3, 1992 |
| 5,430,042 | Lindberg, et al. | Jun. 20, 1991 | Jul. 4, 1995 |
| 5,433,959 | Makino, et al. | Sep. 10, 1993 | Jul. 18, 1995 |
| 5,576,025 | Akiyama, et al. | Mar. 29, 1995 | Nov. 19, 1996 |
| 5,639,478 | Makino, et al. | Jun. 7, 1995 | Jun. 17, 1997 |
| 5,703,110 | Naka, et al. | Sep. 17, 1996 | Dec. 30, 1997 |
| 5,705,517 | Naka, et al. | Oct. 5, 1993 | Jan. 6, 1998 |
| 5,708,017 | Dave, et al. | Apr. 4, 1995 | Jan. 13, 1998 |
| 5,731,006 | Akiyama, et al. | Aug. 20, 1996 | Mar. 24, 1998 |
| 5,824,339 | Shimizu, et al. | Sep. 5, 1996 | Oct. 20, 1998 |
| 5,855,914 | Koyama, et al. | Aug. 9, 1994 | Jan. 5, 1999 |
| 5,879,708 | Makino, et al. | Feb. 27, 1997 | Mar. 9, 1999 |
| 5,948,773 | Akiyama, et al. | May 27, 1997 | Sep. 7, 1999 |
| 6,017,560 | Makino, et al. | Nov. 19, 1998 | Jan. 25, 2000 |
| 6,123,962 | Makino, et al. | Oct. 29, 1999 | Sep. 26, 2000 |
| 6,187,340 | Fakuta, et al. | Sep. 9, 1998 | Feb. 13, 2001 |
| 6,296,875 | Makino, et al. | Jun. 7, 2000 | Oct. 2, 2001 |
| 6,319,904 | Akiyama, et al. | Jul. 7, 1999 | Nov. 20, 2001 |
| 6,328,994 | Shimizu, et al. | May 17, 1999 | Dec. 11, 2001 |
| 4,255,431 | Junggren, et al. | Apr. 5, 1979 | Mar. 10, 1981 |
| 4,508,905 | Junggren, et al. | Apr. 6, 1983 | Apr. 2, 1985 |
| 4,636,499 | Brandstom, et al. | May 30, 1985 | Jan. 13, 1987 |
| 4,738,974 | Brandstrom | Apr. 21, 1986 | Apr. 19, 1988 |
| 5,690,960 | Bengtsson, et al. | Sep. 27, 1994 | Nov. 25, 1997 |
| 5,714,504 | Lindberg, et al. | Jan. 23, 1995 | Feb. 3, 1998 |
| 5,753,265 | Bergstrand, et al. | Jun. 22, 1995 | May 19, 1998 |
| 5,817,338 | Bergstrand, et al. | Jun. 20, 1995 | Oct. 6, 1998 |
| 6,093,734 | Garst, et al. | Aug. 10, 1998 | Jul. 25, 2000 |
| 6,013,281 | Depui, et al. | Mar. 8, 1996 | Jan. 11, 2000 |
| 6,136,344 | Depui, et al. | Apr. 15, 1996 | Oct. 24, 2000 |
| 6,183,776 | Depui, et al. | Feb. 13, 1997 | Feb. 6, 2001 |
| 6,328,994 | Shimizu, et al. | Aug. 4, 1999 | Dec. 11, 2001 |
| 6,479,075 | Odidi, et al. | Jan. 22, 2001 | Nov. 12, 2002 |
| 6,559,167 | Garst et al. | Feb. 14, 2001 | May 6, 2003 |

Examples of salt forms of proton pump inhibiting agents include, for example, a sodium salt form, such as, esomeprazole sodium, omeprazole sodium, rabeprazole sodium, pantoprazole sodium; or a magnesium salt form, such as, esomeprazole magnesium or omeprazole magnesium as described in U.S. Pat. No. 5,900,424; or a calcium salt form; or a potassium salt form, such as, the potassium salt of esomeprazole as described in U.S. patent applicaton Ser. No. 02/0198239, and U.S. Pat. No. 6,511,996. Other salts of esomeprazole are described in U.S. Pat. No. 4,738,974 and U.S. Pat. No. 6,369,085, for example. Pantoprazole and lansoprazole are discussed in U.S. Pat. No. 4,758,579 and in U.S. Pat. No. 4,628,098 respectively.

Included in the methods, kits, combinations and pharmaceutical compositions of the present invention are the tautomers of the described compounds and the pharmaceutically-acceptable salts thereof. Examples of substituted benzimidazole tautomers useful in the present invention, include tautomers of omeprazole, as described in U.S. Pat. Nos. 6,262,085; 6,262,086; 6,268,385; 6,312,723; 6,316,020; 6,326,384; 6,369,087; and 6,444,689; and U.S. patent application Publication Ser. No. 02/0156103, all by Whittle, et al. Examples of isomers of a substituted benzimidazoles useful in the present invention, include an isomer of omeprazole. For example, the compound 5-methoxy-2-[[(4-methoxy-3,5-dimethyl-2-pyridinyl)methyl] sulfinyl]-1H-benzimidazole, having the generic name omeprazole, as well as therapeutically acceptable salts thereof, are described in EP 5129. The single crystal X-ray data and the derived molecular structure of a crystalline form of omeprazole is described by Oishi et al., Acta Cryst. (1989), C45, 1921-1923. This crystal form of omeprazole has been referred to as omeprazole form B. Another crystalline form of omeprazole referred to as omeprazole form A is described in U.S. Pat. No. 6,150,380, and U.S. patent application Publication Ser. No. 02/0156284, by Lovqvist et al. Another crystalline form of omeprazole referred to as omeprazole form C is described in PCT Publication WO 02/085889.

Examples of suitable polymorphs are described in, for example, PCT Publication WO 92/08716; and U.S. Pat. Nos. 4,045,563; 4,182,766; 4,508,905; 4,628,098; 4,636,499; 4,689,333; 4,758,579; 4,783,974; 4,786,505; 4,808,596; 4,853,230; 5,026,560; 5,013,743; 5,035,899; 5,045,321; 5,045,552; 5,093,132; 5,093,342; 5,433,959; 5,464,632; 5,536,735; 5,576,025; 5,599,794; 5,629,305; 5,639,478; 5,690,960; 5,703,110; 5,705,517; 5,714,504; 5,731,006; 5,879,708; 5,900,424; 5,948,773; 5,997,903; 6,017,560; 6,123,962; 6,147,103; 6,150,380; 6,166,213; 6,191,148; 5,187,340; 6,268,385; 6,262,086; 6,262,085; 6,296,875; 6,316,020; 6,328,994; 6,326,384; 6,369,085; 6,369,087; 6,380,234; 6,428,810; 6,444,689; and 6,462,0577.

Also included in the methods, kits, combinations and pharmaceutical compositions of the present invention are the prodrugs of the described compounds and the pharmaceutically-acceptable salts thereof. The term “prodrug” refers to a drug or compound in which the pharmacological action (active curative agent) results from conversion by metabolic processes within the body. Prodrugs are generally considered drug precursors that, following administration to a subject and subsequent absorption, are converted to an active or a more active species via some process, such as a metabolic process. Other products from the conversion process are easily disposed of by the body. Prodrugs generally have a chemical group present on the prodrug which renders it less active and/or confers solubility or some other property to the drug. Once the chemical group has been cleaved from the prodrug the more active drug is generated. Prodrugs may be designed as reversible drug derivatives and utilized as modifiers to enhance drug transport to site-specific tissues. The design of prodrugs to date has been to increase both solid state and aqueous stability, as well as to increase the effective water solubility of the therapeutic compound for targeting to regions where water is the principal solvent. Examples of proton pump inhibiting agents that can be used as prodrugs include, for example, U.S. Pat. No. 6,559,167, which describes prodrugs of proton pump inhibitors using starting materials of, for example, lansoprazole, omeprazole, pantoprazole, and rabeprazole. U.S. Pat. No. 4,686,230 also describes proton pump inhibitors that can act as prodrugs, such as, for example, derivatives of pyridyl methyl sulfinyl benzimidazole compounds. U.S. Pat. No. 5,021,433 also describes pyridyl methyl sulfinyl benzimidazole compounds that can act as prodrugs. N-alkoxycarbonyl benzimidazole derivatives that can act as prodrugs of proton pump inhibiting agents are described in U.S. Pat. No. 4,045,563. Sih., et al., Journal of Medicinal Chemistry, 1991, vol. 34, pp 1049-1062, describe N-acyloxyalkyl, N-alkoxycarbonyl, N-(aminoethyl), and N-alkoxyalkyl derivatives of benzimidazole sulfoxide as prodrugs of proton-pump inhibiting agents. Other examples of prodrugs in general include, for example, Fedorak, et al., Am. J. Physiol, 269:G210-218 (1995), describe dexamethasone-beta-D-glucuronide. McLoed, et al., Gastroenterol., 106:405-413 (1994), describe dexamethasone-succinate-dextrans. Hochhaus, et al., Biomed. Chrom., 6:283-286 (1992), describe dexamethasone-2 1-sulphobenzoate sodium and dexamethasone-21-isonicotinate. Additionally, J. Larsen and H. Bundgaard, Int. J Pharmaceutics, 37, 87 (1987), describe the evaluation of N-acylsulfonamides as potential prodrug derivatives. J. Larsen et al., Int. J Pharmaceutics, 47, 103 (1988), describe the evaluation of N-methylsulfonamides as potential prodrug derivatives. Prodrugs are also described in, for example, Sinkula et al., J. Pharm. Sci., 64:181-210 (1975). A discussion of prodrugs can also be found in T. Higuchi and V. Stella, Pro-drugs as Novel Delivery Systems, Vol. 14 of the A.C.S. Symposium Series. Another discussion of prodrugs can also be found in Edward B. Roche, ed., Bioreversible Carriers in Drug Design, American Pharmaceutical Association and Pergamon Press, 1987.

The term “derivative” refers to a compound that is produced from another compound of similar structure by the replacement of substitution of one atom, molecule or group by another. For example, a hydrogen atom of a compound may be substituted by alkyl, acyl, amino, hydroxyl, halo, haloalkyl, etc., to produce a derivative of that compound.

Other substituted benzimidazole compounds and the salts, hydrates, esters, amides, enantiomers, isomers, tautomers, polymorphs, prodrugs and derivatives thereof may be prepared using standard procedures known to those skilled in the art of synthetic organic chemistry and described, for example, by J. March, Advanced Organic Chemistry; Reactions, Mechanisms and Structure, 4^{th} Ed. (New York: Wiley-Interscience, 1992).

Combinations and mixtures of the above mentioned acid-labile pharmaceutical agents can be used in the methods, kits, combinations, and compositions herein described. Salts, hydrates, esters, amides, enantiomers, isomers, tautomers, polymorphs, prodrugs, and derivatives of the acid-labile pharmaceutical agents may be prepared using standard procedures known to those skilled in the art of synthetic organic chemistry and described, for example, by J. March, Advanced Organic Chemistry; Reactions, Mechanisms and Structure, 4^{th} Ed. (New York: Wiley-Interscience, 1992). For example, acid addition salts are prepared from the free base using conventional methodology, and involves reaction with a suitable acid. Generally, the base form of the drug is dissolved in a polar organic solvent such as methanol or ethanol and the acid is added thereto. The resulting salt either precipitates or may be brought out of solution by addition of a less polar solvent. Suitable acids for preparing acid addition salts include both organic acids, for example, acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, for example, hydrochloric acid, hydrobromic acid, sulfuric acid, nitric acid, phosphoric acid, and the like. An acid addition salt may be reconverted to the free base by treatment with a suitable base. In one embodiment, the acid addition salts of the active agents herein are halide salts, such as may be prepared using hydrochloric or hydrobromic acids. In yet another embodiment, the basic salts here are alkali metal salts, for example, the sodium salt, and copper salts. Preparation of esters involves functionalization of hydroxyl and/or carboxyl groups which may be present within the molecular structure of the drug. The esters are typically acyl-substituted derivatives of free alcohol groups, that is, moieties that are derived from carboxylic acids of the formula RCOOH where R is alkyl, and in one embodiment, is lower alkyl. Esters can be reconverted to the free acids, if desired, by using conventional hydrogenolysis or hydrolysis procedures. Amides may also be prepared using techniques known to those skilled in the art or described in the pertinent literature. For example, amides may be prepared from esters, using suitable amine reactants, or they may be prepared from an anhydride or an acid chloride by reaction with ammonia or a lower alkyl amine.

Unit dosage forms of the compositions of the present invention can typically contain an amount of proton pump inhibiting agent, for example, from about 0.001 parts to about 0.5 parts by weight of the composition, or about 0.01 parts to about 0.4 parts by weight of the composition, or about 0.1 parts to about 0.3 parts by weight of the composition; or between about 1 mg to about 5000 mg, or about 1 mg, or about 2 mg, or about 5 mg, or about 10 mg, or about 15 mg, or about 20 mg, or about 30 mg, or about 40 mg, or about 50 mg, or about 60 mg, or about 70 mg, or about 80, mg, or about 90 mg, or about 100 mg, or about 110 mg, or about 120 mg, or about 130 mg, or about 140 mg, or about 150 mg, or about 160 mg, or about 170 mg, or about 180 mg, or about 190 mg, or about 200 mg, or about 220 mg, or about 240 mg, or about 260 mg, or about 280 mg, or about 300 mg, or about 350, mg, or about 400 mg, or about 450 mg, or about 500 mg, or about 550 mg, or about 600 mg, or about 650 mg, or about 700 mg, or about 750 mg, or about 800 mg, or about 850 mg, or about 900 mg, or about 950 mg, or about 1000 mg, or about 1100 mg, or about 1200 mg, or about 1300 mg, or about 1400 mg, or about 1500, mg, or about 1600 mg, or about 1700 mg, or about 1800 mg, or about 1900 mg, or about 2000 mg, or about 2200 mg, or about 2500 mg, or about 2800 mg, or about 3000 mg, or about 3500 mg, or about 4000 mg, or about 4500 mg, or about 5000 mg of a proton pump inhibiting agent.

Illustratively, for an adult human a unit dosage forms each contain about 0.001 parts, about 0.01 parts, about 0.05 parts, about 0.1 parts, about 0.2 parts, about 0.3 parts, about 0.4 parts, about 0.5 parts, about 0.6 parts, about 0.7 parts, about 0.8 parts, about 1 part, about 1 1 parts, about 1.2 parts, about 1.3 parts, about 1.4 parts, about 1.5 parts, about 1.6 parts, about 1.7 parts, about 1.8 parts, about 1.9 parts, about 2 parts, about 2.1 parts, about 2.2 parts, about 2.3 parts, about 2.4 parts, about 2.5 parts, about 2.6 parts, about 2.7 parts, about 2.8 parts, about 2.9 parts, about 3 parts, about 3.1, about 3.2 parts, about 3.3 parts, about 3.4 parts, about 3.5 parts, about 3.6 parts, about 3.7 parts, about 3.8 parts, about 3.9 parts, about 4, about 4.1, about 4.2 parts, about 4.3 parts, about 4.4 parts, about 4.5 parts, about 4.6 parts, about 4.7 parts, about 4.8 parts, about 4.9 parts, or about 5 parts of proton pump inhibiting agent by weight of the composition.

Illustratively, a unit dosage form may contain about 1 mg, 2 mg, 5 mg, 10 mg, 12.5 mg, 15 mg, 17.5 mg, 20 mg, 22.5 mg, 25 mg, 27.5 mg, 30 mg, 32.5 mg, 35 mg, 37.5 mg, 40 mg, 42.5 mg, 45 mg, 47.5 mg, 50 mg, 52.5 mg, 55 mg, 57.5 mg, 60 mg, 62.5 mg, 65 mg, 67.5 mg, 70 mg, 72.5 mg, 75 mg, 77.5 mg, 80 mg, 82.5 mg, 85 mg, 87.5 mg, 90 mg, 92.5 mg, 95 mg, 97.5 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg 175 mg, 180 mg, 185 mg, 190 mg, 195 mg, 200 mg, 210 mg, 220 mg, 230 mg, 240 mg, 250 mg, 260 mg, 270 mg, 280 mg, 290 mg, 300 mg, 350 mg, 400 mg, or 500 mg of proton pump inhibiting agent.

The dosage unit form can be selected to accommodate the desired frequency of administration (for example, once, twice, three, or four or more times a day) used to achieve the specified daily dosage. The amount of the unit dosage form of the pharmaceutical composition that is administered and the dosage regimen for treating the condition or disorder depends on a variety of factors, including the age, weight, sex and medical condition of the subject, the severity of the condition or disorder, the route and frequency of administration, and thus can vary widely, as is well known. These specific milligram amounts of proton pump inhibiting agent can vary, for example, from between about 0.01% to about 20% or more, depending on the application and desired therapeutic result.

In yet another embodiment of the present invention, the proton pump inhibiting agent is present in the composition in an amount of about 0.05% to about 90% by weight of the composition. Illustratively, the percent of the proton pump inhibiting agent is about 0.05%, or about 0.1%, or about 0.2%, or about 0.3%, or about 0.4%, or about 0.5%, or about 0.6%, or about 0.7%, or about 0.8%, or about 0.9%, or about I %, or about 2%, or about 3%, or about 4%, or about 5%, or about 6%, or about 7%, or about 8%, or about 9%, or about 10%, or about 15%, or about 20%, or about 25%, or about 30%, or about 35%, or about 40%, or about 45%, or about 50%, or about 55%, or about 60%, or about 65%, or about 70%, or about 75%, or about 80%, or about 85%, or about 90% by weight of the composition. These specific percentages of proton pump inhibiting agent can vary, for example, from between about 0.01% to about 20% or more, depending on the application and desired therapeutic result.

In the written descriptions of molecules and groups, molecular descriptors can be combined to produce words or phrases that describe structural groups or are combined to describe structural groups. Such descriptors are used in this document. Common illustrative examples include such terms as aralkyl (or arylalkyl), heteroaralkyl, heterocycloalkyl, cycloalkylalkyl, aralkoxyalkoxycarbonyl, and the like. A specific example of a compound encompassed with the latter descriptor aralkoxyalkoxycarbonyl is C₆H₅—CH₂—CH₂—O—CH₂—O—(C═O)— wherein C₆H₅— is phenyl. It is also to be noted that a structural group can have more than one descriptive word or phrase in the art, for example, heteroaryloxyalkylcarbonyl can also be termed heteroaryloxyalkanoyl. Such combinations are used herein in the description of the processes, compounds and compositions of this invention and further examples are described below. The following list is not intended to be exhaustive or drawn out but provide illustrative examples of words or phrases (terms) that are used herein.

As utilized herein, the term “alkyl,” alone or in combination, means a straight-chain or branched-chain alkyl radical containing one to about twelve carbon atoms, preferably one to about ten carbon atoms, and more preferably one to about six carbon atoms. Examples of such radicals include methyl, ethyl, n-propyl, isopropyl, n-butyl, isobutyl, sec-butyl, tert-butyl, pentyl, iso-amyl, hexyl, octyl, and the like.

The term “acyl,” alone or in combination, means a radical provided by the residue after removal of hydroxyl from an organic acid. Examples of such acyl radicals include alkanoyl and aroyl radicals. Examples of such alkanoyl radicals include formyl, acetyl, propionyl, butyryl, isobutyryl, valeryl, isovaleryl, pivaloyl, hexanoyl, trifluoroacetyl, and the like.

The term “carbonyl” or “oxo,” alone or in combination, that is, used with other terms, such as “alkoxycarbonyl,” means a —C(═O)— group wherein the remaining two bonds (valences) can be independently substituted. The term carbonyl is also intended to encompass a hydrated carbonyl group —C(OH)₂—.

The term “hydrido,” or “hydrogen,” alone or in combination, means a single hydrogen atom (H). This hydrido radical may be attached, for example, to an oxygen atom to form a hydroxyl radical or two hydrido radicals may be attached to a carbon atom to form a methylene (—CH₂—) radical.

The term “halo,” or “halogen,” alone or in combination, means halogen such as fluoride, chloride, bromide or iodide.

The term “haloalkyl”, alone or in combination, means an alkyl radical having the significance as defined above wherein one or more hydrogens are replaced with a halogen. Specifically embraced are monohaloalkyl, dihaloalkyl and polyhaloalkyl radicals. A monohaloalkyl radical, for one example, may have either an iodo, bromo, chloro or fluoro atom within the radical. Dihalo and polyhaloalkyl radicals may have two or more of the same halo atoms or a combination of different halo radicals.

The term “thiol” or “sulfhydryl,” alone or in combination, means a —SH group. The term “thio” or “thia,” alone or in combination, means a thiaether group; that is, an ether group wherein the ether oxygen is replaced by a sulfur atom.

The term “amino,” alone or in combination, means an amine or —NH₂ group whereas the term mono-substituted amino, alone or in combination, means a substituted amine —N(H)(substituent) group wherein one hydrogen atom is replaced with a substituent, and disubstituted amine means a —N(substituent)₂ wherein two hydrogen atoms of the amino group are replaced with independently selected substituent groups.

Amines, amino groups and amides are compounds that can be designated as primary (I°), secondary (II°) or tertiary (III°) or unsubstituted, mono-substituted or N,N-disubstituted depending on the degree of substitution of the amino nitrogen. Quaternary amine (ammonium)(IV°) means a nitrogen with four substituents [—N⁺(substituent)₄] that is positively charged and accompanied by a counter ion, whereas N-oxide means one substituent is oxygen and the group is represented as [—N⁺(substituent)₃—O⁻]; that is, the charges are internally compensated.

The term “cyano,” alone or in combination, means a —C-triple bond-N (—C≡N) group.

The term “hydroxyl,” alone or in combination, means a —OH group.

The term “nitro,” alone or in combination, means a —NO₂ group.

The term “sulfonyl,” alone or in combination, that is, linked to other terms such as alkylsulfonyl, means a —SO₂— group wherein the depicted remaining two bonds (valences) can be independently substituted.

The term “sulfoxido,” alone or in combination, means a —SO— group wherein the remaining two bonds (valences) can be independently substituted.

The term “sulfone,” alone or in combination, means a —SO₂— group wherein the depicted remaining two bonds (valences) can be independently substituted.

The term “alkylthio,” alone or in combination, means a radical containing a linear or branched alkyl radical, of one to about ten carbon atoms attached to a divalent sulfur atom. Illustratively, alkylthio radicals are radicals having alkyl radicals of one to six carbon atoms. Examples of such alkylthio radicals are methylthio, ethylthio, propylthio, butylthio and hexylthio.

The term “heterocyclo” embraces saturated, partially unsaturated and unsaturated heteroatom-containing ring-shaped radicals, where the heteroatoms may be selected from nitrogen, sulfur and oxygen. Examples of saturated heterocyclo radicals include saturated three- to six-membered heteromonocylic group containing one to four nitrogen atoms (for example pyrrolidinyl, imidazolidinyl, piperidino, piperazinyl, etc.); saturated three-to six-membered heteromonocyclic group containing one to two oxygen atoms and one to three nitrogen atoms (for example morpholinyl, etc.); saturated three-to six-membered heteromonocyclic group containing one to two sulfur atoms and one to three nitrogen atoms (for example, thiazolidinyl, etc.).

Heterocyclo compounds include benzofused heterocyclic compounds such as benzo-1,4-dioxane. Such a moiety can be optionally substituted on one or more ring carbon atoms by halogen, hydroxy, hydroxycarbonyl, alkyl, alkoxy, oxo, and the like, and/or on a secondary nitrogen atom (that is, —NH—) of the ring by alkyl, aralkoxycarbonyl, alkanoyl, aryl or arylalkyl or on a tertiary nitrogen atom (that is, ═N—) by oxido and that is attached via a carbon atom. The tertiary nitrogen atom with three substituents can also attached to form a N-oxide [═N(O)—] group.

The term “aryl,” alone or in combination, means a five-or six-membered carbocyclic aromatic ring-containing moiety or a five-or six-membered carbocyclic aromatic system containing two or three rings wherein such rings are attached together in a pendent manner, or a fused ring system containing two or three rings that have all carbon atoms in the ring; that is, a carbocyclic aryl radical. Aryl moieties may also be substituted with one or more substituents including alkyl, alkoxyalkyl, alkylaminoalkyl, carboxyalkyl, alkoxycarbonylalkyl, aminocarbonylalkyl, alkoxy, aralkoxy, hydroxyl, amino, halo, nitro, alkylamino, acyl, cyano, carboxy, aminocarbonyl, alkoxycarbonyl and aralkoxycarbonyl.

The term “heteroaryl,” alone or in combination means a five-or six-membered aromatic ring-containing moiety or a fused ring system (radical) containing two or three rings that have carbon atoms and also one or more heteroatoms in the ring(s) such as sulfur, oxygen and nitrogen. Examples of such heterocyclic or heteroaryl groups are pyrrolidinyl, piperidyl, piperazinyl, morpholinyl, thiamorpholinyl, pyrrolyl, imidazolyl (for example, imidazol-4-yl, 1-benzyloxycarbonylimidazol-4-yl, and the like), pyrazolyl, pyridyl, pyrazinyl, pyrimidinyl, furyl, tetrahydrofuryl, thienyl, triazolyl, tetrazolyl, oxazolyl, oxadiazoyl, thiazolyl, thiadiazoyl, indolyl (for example, 2-indolyl, and the like), quinolinyl, (for example, 2-quinolinyl, 3-quinolinyl, 1-oxido-2-quinolinyl, and the like), isoquinolinyl (for example, 1-isoquinolinyl, 3-isoquinolinyl, and the like), tetrahydroquinolinyl (for example, 1,2,3,4-tetrahydro-2-quinolyl, and the like), 1,2,3,4-tetrahydroisoquinolinyl (for example, 1,2,3,4-tetrahydro-1-oxo-isoquinolinyl, and the like), quinoxalinyl, β-carbolinyl, 2-benzofurancarbonyl, benzothiophenyl, 1-, 2-, 4- or 5-benzimidazolyl, and the like radicals.

The term “pharmaceutically acceptable” is used adjectivally herein to mean that the modified noun is appropriate for use in a pharmaceutical product. Pharmaceutically acceptable cations include metallic ions and organic ions. Illustratively, metallic ions include, but are not limited to appropriate alkali metal (Group Ia) salts, alkaline earth metal (Group IIa) salts and other physiological acceptable metal ions. Exemplary ions include aluminum, calcium, lithium, magnesium, potassium, sodium and zinc in their usual valences. Preferred organic ions include protonated tertiary amines and quaternary ammonium cations, including in part, trimethylamine, diethylamine, N,N′-dibenzylethylenediamine, chloroprocaine, choline, diethanolamine, ethylenediamine, meglumine (N-methylglucamine) and procaine. Exemplary pharmaceutically acceptable acids include without limitation hydrochloric acid, hydrobromic acid, phosphoric acid, sulfuric acid, methanesulfonic acid, acetic acid, formic acid, tartaric acid, maleic acid, malic acid, citric acid, isocitric acid, succinic acid, lactic acid, gluconic acid, glucuronic acid, pyruvic acid oxalacetic acid, fumaric acid, propionic acid, aspartic acid, glutamic acid, benzoic acid, and the like.

The acid-labile pharmaceutical agents of the present invention may be in the form of granules, spheroids, microspheres, seeds, pellets, beads, microcapsules, agglomerates, mini-tablets, tablets, or other multi-particulate forms manufactured by conventional pharmacological techniques.

A class of buffering agents useful in the methods, kits, combinations, and compositions of the present invention include an agent possessing pharmacological activity as a weak base or a strong base. In one embodiment, the buffering agent when formulated or delivered (for example, before, during and/or after) with an acid-labile pharmaceutical agent functions to substantially prevent or inhibit the acid degradation of the acid-labile pharmaceutical agent by gastrointestinal fluid for a period of time, for example, for a period of time sufficient to preserve the bioavailability of the acid-labile pharmaceutical agent administered. In one aspect of the present invention, the buffering agent employed in the present invention includes a salt of a Group IA metal, including, for example a bicarbonate salt of a Group IA metal, a carbonate salt of a Group IA metal, an alkali earth metal buffering agent, an aluminum buffering agent, a calcium buffering agent, or a magnesium buffering agent. Other suitable buffering agents include alkali (sodium and potassium) or alkali earth (calcium and magnesium) carbonates, phosphates, bicarbonates, citrates, borates, acetates, phthalates, tartrates, succinates and the like, such as, for example, sodium or potassium phosphate, citrate, borate, acetate, bicarbonate and carbonate. Illustratively, a buffering agent of interest that can be used in the methods, kits, combinations, and compositions of the present invention includes, but is not limited to, an amino acid, an acid salt of an amino acid, an alkali salt of an amino acid, aluminum hydroxide, aluminum hydroxide/magnesium carbonate, aluminum hydroxide/magnesium carbonate/calcium carbonate co-precipitate, aluminum magnesium hydroxide, aluminum hydroxide/magnesium hydroxide co-precipitate, aluminum hydroxide/sodium bicarbonate coprecipitate, aluminum glycinate, calcium acetate, calcium bicarbonate, calcium borate, calcium carbonate, calcium citrate, calcium chloride, calcium gluconate, calcium glycerophosphate, calcium hydroxide, calcium lactate, calcium phthalate, calcium phosphate, calcium succinate, calcium tartrate, dibasic sodium phosphate, dipotassium hydrogen phosphate, dipotassium phosphate, disodium hydrogen phosphate, disodium succinate, dry aluminum hydroxide gel, L-arginine, magnesium acetate, magnesium aluminate, magnesium borate, magnesium bicarbonate, magnesium carbonate, magnesium citrate, magnesium gluconate, magnesium hydroxide, magnesium lactate, magnesium metasilicate aluminate, magnesium oxide, magnesium phthalate, magnesium phosphate, magnesium silicate, magnesium succinate, magnesium tartrate, potassium acetate, potassium carbonate, potassium bicarbonate, potassium borate, potassium citrate, potassium metaphosphate, potassium phthalate, potassium phosphate, potassium polyphosphate, potassium pyrophosphate, potassium succinate, potassium tartrate, sodium acetate, sodium bicarbonate, sodium borate, sodium carbonate, sodium citrate, sodium gluconate, sodium dihydrogen phosphate, sodium hydrogen phosphate, sodium hydroxide, sodium lactate, sodium phthalate, sodium phosphate, sodium polyphosphate, sodium pyrophosphate, sodium sesquicarbonate, sodium succinate, sodium tartrate, sodium tripolyphosphate, synthetic hydrotalcite, tetrapotassium pyrophosphate, tetrasodium pyrophosphate, trihydroxymethylaminomethane, tripotassium phosphate, trisodium phosphate, and trometamol. (Based in part upon the list provided in The Merck Index, Merck & Co. Rahway, N. J. (2001)). In addition, proteins or protein hydrolysates can serve as buffering agents owing to their ability to rapidly neutralize acid. Combinations of the above mentioned buffering agents can be used in the methods, kits, combinations, and compositions herein described.

In another embodiment, the buffering agent is present in the methods, kits, combinations, and compositions of the present invention in an amount of about 0.05 mEq to about 15 mEq/mg of acid-labile pharmaceutical agent, for example, a proton pump inhibiting agent. In another embodiment of the present invention the buffering agent is present in an amount of about 0.1 mEq to about 10 mEq/mg of proton pump inhibiting agent. In another embodiment of the present invention the buffering agent is present in an amount of about 0.1 mEq to about 5 mEq/mg of proton pump inhibiting agent. In another embodiment of the present invention the buffering agent is present in an amount of about 0.2 mEq to about 2.5 mEq/mg of proton pump inhibiting agent. In yet another embodiment of the present invention the buffering agent is present in an amount of at least about 0.5 mEq/mg proton pump inhibitor, or at least about 1 mEq/mg proton pump inhibitor, or at least about 2 mEq/mg proton pump inhibitor, or at least about 4 mEq/mg proton pump inhibitor, or at least about 5 mEq/mg proton pump inhibitor, or at least about 7.5 mEq/mg proton pump inhibitor, or at least about 10 mEq/mg proton pump inhibitor, or at least about 15 mEq/mg proton pump inhibitor.

In yet another embodiment of the present invention the total amount of buffering agent present in the pharmaceutical composition is about 2 mEq to about 160 mEq. In still another embodiment, the buffering agent is present in an amount of about 5 mEq to about 120 mEq. In still another embodiment, the buffering agent is present in an amount of about 10 mEq to about 70 mEq. In still another embodiment, the buffering agent is present in an amount of about 15 mEq to about 55 mEq. In still another embodiment, the buffering agent is present in an amount of about 20 mEq to about 40 mEq. In still another embodiment, the buffering agent is present in an amount of about 12.5 mEq to about 30 mEq. Illustratively, the total amount of buffering agent in a composition of the present invention is about 0.1 mEq, or about 0.2 mEq, or about 0.5 mEq, or about 1 mEq, or about 2 mEq, or about 3 mEq, or about 4 mEq, or about 5 mEq, or about 7.5 mEq, or about 10 mEq, or about 12.5 mEq, or about 15 mEq, or about 16 mEq, or about 17.5 mEq, or about 20 mEq, or about 22.5 mEq, or about 25 mEq, or about 27.5 mEq, or about 30 mEq, or about 32.5 mEq, or about 35 mEq, or about 37.5 mEq, or about 40 mEq, or about 42.5 mEq, or about 45 mEq, or about 47.5 mEq, or about 50 mEq, or about 52.5 mEq, or about 55 mEq, or about 57.5 mEq, or about 60 mEq, or about 62.5 mEq, or about 65 mEq, or about 67.5 mEq, or about 70 mEq, or about 75 mEq, or about 80 mEq, or about 85 mEq, or about 90 mEq, or about 95 mEq, or about 100 mEq, or about 110 mEq, or about 120 mEq, or about 130 mEq, or about 140 mEq, or about 150 mEq, or about 160 mEq. These specific mEq amounts of buffering agent can vary, for example, from between about 0.01% to about 20% or more, depending on the application and desired therapeutic result.

In yet another embodiment of the present invention, the amount of the buffering agent is more than about 5 times the amount of the proton pump inhibiting agent on a weight to weight basis in the composition. In yet another embodiment of the present invention, the amount of the buffering agent is more than about 10 to about 100 times the amount of the proton pump inhibiting agent on a weight to weight basis in the composition. In yet another embodiment of the present invention, the amount of the buffering agent is present in an amount more than about 5 times, or more than about 10 times, or more than about 20 times, or more than about 30 times, or more than about 40 times, more than about 50 times, or more than about 60 times, or more than about 70 times, or more than about 80 times, or more than about 90 times, or more than about 100 times the amount of the proton pump inhibiting agent on a weight to weight basis in the composition.

In one embodiment of the present invention, the buffering agent is sodium bicarbonate, sodium carbonate, calcium carbonate, calcium bicarbonate, magnesium oxide, magnesium hydroxide, or mixtures thereof, and is present in the methods, kits, combinations and compositions in an amount of at least about 250 mg. In another embodiment, the sodium bicarbonate, sodium carbonate, calcium carbonate, calcium bicarbonate, or mixtures thereof is present in an amount of at least about 400 mg. In yet another embodiment, the sodium bicarbonate, sodium carbonate, calcium carbonate, calcium bicarbonate, or mixtures thereof is present in an amount from about 250 mg to about 4000 mg. In still another embodiment, the sodium bicarbonate, sodium carbonate, calcium carbonate, calcium bicarbonate, or mixtures thereof is present in an amount from about 1000 mg to about 2000 mg. And in still another embodiment, the sodium bicarbonate, sodium carbonate, calcium carbonate, calcium bicarbonate, or mixtures thereof is present in an amount from about 1250 mg to about 1750 mg. In still another embodiment, the sodium bicarbonate, sodium carbonate, calcium carbonate, calcium bicarbonate, or mixtures thereof is present in an amount from about 500 mg to about 1680 mg.

Illustratively, the amount of buffering agent or agents in a composition of the present invention is about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1620 mg, about 1640 mg, about 1660 mg, about 1680 mg, about 1700 mg, about 1725 mg, about 1750 mg, about 1800 mg, about 1825 mg, about 1850 mg, about 1875 mg, about 1900 mg, about 1950 mg, or about 2000 mg, or about 2500 mg, or about 3000 mg, or about 3500 mg. These specific amounts can vary, for example, from between about 0.01% to about 20% or more, depending on the application and desired therapeutic result.

In one embodiment of the present invention, the buffering agent is sodium bicarbonate and is present in the methods, kits, combinations and compositions in an amount of at least about 250 mg. In another embodiment, the sodium bicarbonate is present in an amount of at least about 400 mg. In yet another embodiment, the sodium bicarbonate is present in an amount from about 250 mg to about 4000 mg. In still another embodiment, the sodium bicarbonate is present in an amount from about 1000 mg to about 2000 mg. And in still another embodiment, the sodium bicarbonate is present in an amount from about 1250 mg to about 1750 mg. In still another embodiment, the sodium bicarbonate is present in an amount from about 500 mg to about 1680 mg. Illustratively, the amount of sodium bicarbonate in a composition of the present invention is about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1620 mg, about 1640 mg, about 1660 mg, about 1680 mg, about 1700 mg, about 1725 mg, about 1750 mg, about 1800 mg, about 1825 mg, about 1850 mg, about 1875 mg, about 1900 mg, about 1950 mg, or about 2000 mg. These specific amounts can vary, for example, from between about 0.01% to about 20% or more, depending on the application and desired therapeutic result.

In one embodiment of the present invention, the buffering agent is sodium carbonate and is present in the methods, kits, combinations and compositions in an amount of at least about 250 mg. In another embodiment, the sodium carbonate is present in an amount of at least about 400 mg. In yet another embodiment, the sodium carbonate is present in an amount from about 250 mg to about 4000 mg. In still another embodiment, the sodium carbonate is present in an amount from about 1000 mg to about 2000 mg. And in still another embodiment, the sodium carbonate is present in an amount from about 1250 mg to about 1750 mg. In still another embodiment, the sodium carbonate is present in an amount from about 500 mg to about 1680 mg. Illustratively, the amount of sodium carbonate in a composition of the present invention is about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1620 mg, about 1640 mg, about 1660 mg, about 1680 mg, about 1700 mg, about 1725 mg, about 1750 mg, about 1800 mg, about 1825 mg, about 1850 mg, about 1875 mg, about 1900 mg, about 1950 mg, or about 2000 mg. These specific amounts can vary, for example, from between about 0.01% to about 20% or more, depending on the application and desired therapeutic result.

In one embodiment of the present invention, the buffering agent is calcium carbonate and is present in the methods, kits, combinations and compositions in an amount of at least about 250 mg. In another embodiment, the calcium carbonate is present in an amount of at least about 400 mg. In yet another embodiment, the calcium carbonate is present in an amount from about 250 mg to about 4000 mg. In still another embodiment, the calcium carbonate is present in an amount from about 1000 mg to about 2000 mg. And in still another embodiment, the calcium carbonate is present in an amount from about 1250 mg to about 1750 mg. In still another embodiment, the calcium carbonate is present in an amount from about 500 mg to about 1680 mg. Illustratively, the amount of calcium carbonate in a composition of the present invention is about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1620 mg, about 1640 mg, about 1660 mg, about 1680 mg, about 1700 mg, about 1725 mg, about 1750 mg, about 1800 mg, about 1825 mg, about 1850 mg, about 1875 mg, about 1900 mg, about 1950 mg, or about 2000 mg. These specific amounts can vary, for example, from between about 0.01% to about 20% or more, depending on the application and desired therapeutic result.

In one embodiment of the present invention, the buffering agent is calcium bicarbonate and is present in the methods, kits, combinations and compositions in an amount of at least about 250 mg. In another embodiment, the calcium bicarbonate is present in an amount of at least about 400 mg. In yet another embodiment, the calcium bicarbonate is present in an amount from about 250 mg to about 4000 mg. In still another embodiment, the calcium bicarbonate is present in an amount from about 1000 mg to about 2000 mg. And in still another embodiment, the calcium bicarbonate is present in an amount from about 1250 mg to about 1750 mg. In still another embodiment, the calcium bicarbonate is present in an amount from about 500 mg to about 1680 mg. Illustratively, the amount of calcium bicarbonate in a composition of the present invention is about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1620 mg, about 1640 mg, about 1660 mg, about 1680 mg, about 1700 mg, about 1725 mg, about 1750 mg, about 1800 mg, about 1825 mg, about 1850 mg, about 1875 mg, about 1900 mg, about 1950 mg, or about 2000 mg. These specific amounts can vary, for example, from between about 0.01% to about 20% or more, depending on the application and desired therapeutic result.

In one embodiment of the present invention, the buffering agent is sodium bicarbonate and sodium carbonate and are present in the methods, kits, combinations and compositions in an amount of at least about 250 mg. In another embodiment, the sodium bicarbonate and sodium carbonate are present in an amount of at least about 400 mg. In yet another embodiment, the sodium bicarbonate and sodium carbonate are present in an amount from about 250 mg to about 4000 mg. In still another embodiment, the sodium bicarbonate and sodium carbonate are present in an amount from about 1000 mg to about 2000 mg. And in still another embodiment, the sodium bicarbonate and sodium carbonate are present in an amount from about 1250 mg to about 1750 mg. In still another embodiment, the sodium bicarbonate and sodium carbonate are present in an amount from about 500 mg to about 1680 mg. Illustratively, the amount of sodium bicarbonate and sodium carbonate in a composition of the present invention is about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1620 mg, about 1640 mg, about 1660 mg, about 1680 mg, about 1700 mg, about 1725 mg, about 1750 mg, about 1800 mg, about 1825 mg, about 1850 mg, about 1875 mg, about 1900 mg, about 1950 mg, or about 2000 mg. These specific amounts can vary, for example, from between about 0.01% to about 20% or more, depending on the application and desired therapeutic result.

In one embodiment of the present invention, the buffering agent is sodium bicarbonate and calcium carbonate and are present in the methods, kits, combinations and compositions in an amount of at least about 250 mg. In another embodiment, the sodium bicarbonate and calcium carbonate are present in an amount of at least about 400 mg. In yet another embodiment, the sodium bicarbonate and calcium carbonate are present in an amount from about 250 mg to about 4000 mg. In still another embodiment, the sodium bicarbonate and calcium carbonate are present in an amount from about 1000 mg to about 2000 mg. And in still another embodiment, the sodium bicarbonate and calcium carbonate are present in an amount from about 1250 mg to about 1750 mg. In still another embodiment, the sodium bicarbonate and calcium carbonate are present in an amount from about 500 mg to about 1680 mg. Illustratively, the amount of sodium bicarbonate and calcium carbonate in a composition of the present invention is about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1620 mg, about 1640 mg, about 1660 mg, about 1680 mg, about 1700 mg, about 1725 mg, about 1750 mg, about 1800 mg, about 1825 mg, about 1850 mg, about 1875 mg, about 1900 mg, about 1950 mg, or about 2000 mg. These specific amounts can vary, for example, from between about 0.01% to about 20% or more, depending on the application and desired therapeutic result.

In one embodiment of the present invention, the buffering agent is calcium carbonate and sodium carbonate and are present in the methods, kits, combinations and compositions in an amount of at least about 250 mg. In another embodiment, the calcium carbonate and sodium carbonate are present in an amount of at least about 400 mg. In yet another embodiment, the calcium carbonate and sodium carbonate are present in an amount from about 250 mg to about 4000 mg. In still another embodiment, the calcium carbonate and sodium carbonate are present in an amount from about 1000 mg to about 2000 mg. And in still another embodiment, the calcium carbonate and sodium carbonate are present in an amount from about 1250 mg to about 1750 mg. In still another embodiment, the calcium carbonate and sodium carbonate are present in an amount from about 500 mg to about 1680 mg. Illustratively, the amount of calcium carbonate and sodium carbonate in a composition of the present invention is about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1620 mg, about 1640 mg, about 1660 mg, about 1680 mg, about 1700 mg, about 1725 mg, about 1750 mg, about 1800 mg, about 1825 mg, about 1850 mg, about 1875 mg, about 1900 mg, about 1950 mg, or about 2000 mg. These specific amounts can vary, for example, from between about 0.01% to about 20% or more, depending on the application and desired therapeutic result.

For oral administration, the pharmaceutical composition of the present invention can contain a desired amount of acid-labile pharmaceutical agent and/or buffering agent and be in the form of, for example, a tablet (for example, suspension tablet, a bite suspension tablet, a rapid dispersion tablet, a chewable tablet, an effervescent tablet, a bilayer tablet, and a tablet-in-a-tablet), pill, powder (for example, a packaged powder, a dispensable powder, an effervescent powder), capsule (for example, a soft or a hard gelatin capsule), lozenge, sachet, cachet, troche, pellet, granule, aerosol (as a solid or in a liquid medium), or any other form reasonably adapted for oral administration. Such a pharmaceutical composition can be made in the form of a discrete dosage unit containing a predetermined amount of acid-labile pharmaceutical agent and buffering agent, such as tablets or capsules.

In one embodiment of the present invention, the controlled-release component contains about 1 mg to about 500 mgs of an acid-labile pharmaceutical agent, for example, a proton pump inhibiting agent. In another embodiment, the controlled-release component contains about 5 mg to about 240 mg of a proton pump inhibiting agent. In another embodiment the controlled-release component contains about 10 mg to about 120 mg of a proton pump inhibiting agent. In yet another embodiment the controlled-release component contains about 15 mg to about 80 mg of a proton pump inhibiting agent. In yet another embodiment the controlled-release component contains about 20 mg to about 60 mg of a proton pump inhibiting agent. In another embodiment the controlled-release component contains about 30 mg to about 40 mg of a proton pump inhibiting agent. Additionally, these illustrated amounts can vary, for example, from between about 0.01% to about 20% or more, depending on the application and desired therapeutic result.

It will be understood that the amount of proton pump inhibiting agent that is administered to a subject is dependent on, for example, the type of subject, the sex, age, general health, diet, and/or body weight of the subject. Illustratively, where the agent is a substituted benzimidazole such as, for example, omeprazole, tenatoprazole, lansoprazole, pantoprazole, rabeprazole, esomeprazole, pariprazole, or leminoprazole, and the subject is, for example, a child or a small animal (for example, a dog), a relatively low amount of the agent in the dose range of about 1 mg to about 20 mg is likely to provide blood serum concentrations consistent with therapeutic effectiveness. For even smaller mammals, such as, for example, a guinea pig, even smaller amounts of the agent are required. Where the subject is an adult human or a large animal (for example, a horse), achievement of therapeutic blood serum concentrations of the agent are likely to require dose units containing a relatively greater amount of the agent, for example, about a 15 mg, 20 mg, 30 mg, 40 mg, 50 mg, 60 mg, 70 mg, 80 mg, 90 mg, 100 mg, 110 mg, or 120 mg dose for an adult human, or about a 150 mg, 200 mg, 400 mg, 800 mg, 1000 mg, 2000 mg, 3000 mg, 4000 mg, or 5000 mg (or more) dose for an adult horse.

In yet another embodiment, the composition of the present invention contains about 0.1 mg/kg to about 10 mg/kg, or about 0.01 mg/kg to about 5 mg/kg, or about 0.5 mg/kg to about 10 mg/kg, or about 0.5 mg/kg to about 5 mg/kg, or about 0.5 mg/kg to about 2.5 mg/kg of an acid-labile pharmaceutical agent, for example, a proton pump inhibiting agent, per kilogram of body weight per dose. Illustratively, the amount of the acid-labile pharmaceutical agent, for example, a proton pump inhibiting agent, present in a composition of the present invention is about 0.1 mg/kg, or about 0.5 mg/kg, or about 1 mg/kg, or about 1.5 mg/kg, or about 2 mg/kg, or about 2.5 mg/kg, or about 3 mg/kg, or about 3.5 mg/kg, or about 4 mg/kg, or about 4.5 mg/kg, or about 5 mg/kg of an acid-labile pharmaceutical agent, for example, a proton pump inhibiting agent, per kilogram of body weight per dose. Additionally, these illustrated amounts can vary, for example, from between about 0.01% to about 20% or more, depending on the application and desired therapeutic result. The above dose may be administered once or in several divided doses per day.

The solid compositions of the present invention are generally in the form of discrete unit dosage forms, such as in a tablet (for example, a suspension tablet, a bite suspension tablet, a rapid dispersion tablet, a chewable tablet, or an effervescent tablet), pill, powder (for example, a packaged powder, a dispensable powder, an effervescent powder), capsule (for example, a soft or a hard gelatin capsule), lozenge, sachet, cachet, troche, pellet, or granule. Such dosage units may be given at least once, twice, three, or four times a day, or as many times as needed to elicit a therapeutic response. A particular unit dosage form can be selected to accommodate the desired frequency of administration used to achieve a specified daily dosage.

Illustratively, the approximate adult daily oral dosage is typically 20 mg to 40 mg of omeprazole, 15 mg to 30 mg lansoprazole, 20 mg to 40 mg pantoprazole, 20 mg rabeprazole, 20 to 40 mg esomeprazole, and the pharmacologically equivalent doses of pariprazole and leminoprazole. See, Physicians' Desk Reference, 55^{th} Edition, 2001.

A composition of the present invention can be administered orally or enterally to a subject. This can be accomplished, for example, by administering a suspension of the present invention via a nasogastric tube or other indwelling tube placed in the gastrointestinal tract. In one embodiment of the present invention, in order to avoid the disadvantages associated with administering large amounts of sodium bicarbonate, the proton pump inhibiting agent of the present invention is administered in a single dose which does not require any further administration of bicarbonate, or other buffering agent following the administration of the proton pump inhibiting agent, nor does it require a large amount of bicarbonate or buffering agent in total. The present invention eliminates the need to pre-or post-dose with additional volumes of water and sodium bicarbonate. The amount of bicarbonate administered via the single dose administration of the present invention is less than the amount of bicarbonate administered as taught in the references cited above.

The term “immediate-release” is intended to refer to a formulation in which release of the agent is substantially instantaneous (for example, within about 30 seconds to about 60 minutes or less) into an aqueous media, such as gastrointestinal fluid, including, for example, the gastrointestinal content of the stomach, the saliva content of the mouth after oral administration, or a 1% sodium dodecyl sulfate solution, or 0.1N hydrochloric acid in water at 37° C. With an “immediate-release” formulation, oral administration results in immediate-release of the agent from the composition into gastrointestinal fluid. For controlled-release formulations, the opposite is generally true, the rate of release of drug from the dosage form is the rate-limiting step in the delivery of the drug to the target area. The term “controlled release” includes any nonimmediate-release formulation, including but not limited to, enteric coated formulations, which release the agent from the composition into gastrointestinal fluid having an appropriate pH within about 1 minute to about 90 minutes, or within about 1 minute, or within about 5 minutes, or within about 10 minutes, or within about 15 minutes, or within about 20 minutes, or within about 25 minutes, or within about 30 minutes, or within about 35 minutes, or within about 40 minutes, or within about 45 minutes, or within about 50 minutes, or within about 55 minutes, or within about 60 minutes, or within about 65 minutes, or within about 70 minutes, or within about 75 minutes, or within about 80 minutes, or within about 85 minutes, or within about 90 minutes after contacting the fluid. See also, Remington: The Science and Practice of Pharmacy, Nineteenth Ed. (Easton, Pa.: Mack Publishing Company, 1995).

“Plasma concentration” refers to the concentration of a substance in blood plasma or blood serum of a subject.

“Drug absorption” or “absorption” refers to the process of movement from the site of administration of a drug toward the systemic circulation, for example, into the bloodstream of a subject.

“Bioavailability” refers to the extent to which an active moiety (drug or metabolite) is absorbed into the general circulation and becomes available at the site of drug action in the body.

“Metabolism” refers to the process of chemical alteration of drugs in the body.

“Half-life” refers to the time required for the plasma drug concentration or the amount in the body to decrease by 50% from its maximum concentration.

The use of the term “about” in the present disclosure means “approximately,” and illustratively, the use of the term “about” indicates that values slightly outside the cited values (for example, plus or minus 0.1% to 20%) may also be effective and safe, and such dosages are also encompassed by the scope of the present claims.

The term “measurable serum concentration” means the serum concentration (typically measured in mg, μg, or ng of therapeutic agent per ml, dl, or I of blood serum) of a therapeutic agent absorbed into the bloodstream after administration. Illustratively, the serum concentration of a proton pump inhibiting agent of the present invention that corresponds to a measurable serum concentration for an adult subject is greater than about 5 ng/ml. In another embodiment of the present invention, the serum concentration of the proton pump inhibiting agent that corresponds to a measurable serum concentration for an adult human is less than about 10.0 μg/ml. In yet another embodiment of the present invention, the serum concentration of the proton pump inhibiting agent that corresponds to a measurable serum concentration for an adult human is from about 0.01 μg/ml to about 5 μg/ml.

In attempting to provide for rapid release of an acid-labile pharmaceutical agent from a dosage form covered, coated or layered with an enteric coating, Applicants found that use of an enteric coated dosage form as generally practiced in the art did not provide effective release of the agent into gastrointestinal fluid at the desired location in the gastrointestinal tract when administered to a subject and/or under certain pH conditions. Such enteric coated dosage forms are, for example, generally described in the patents listed in Table No. 2.

TABLE NO. 2
Issued U.S. Patents Teaching Enteric Coated
Proton Pump Inhibiting Agents

| U.S. | | | |
|---|---|---|---|
| Pat. No. | Inventor | Filing Date | Issue Date |
| 4,628,098 | Nohara, et al. | Jul. 29 1985 | Dec. 9, 1986 |
| 4,689,333 | Nohara, et al. | Dec. 2, 1986 | Aug. 25, 1987 |
| 4,786,505 | Lovgren, et al. | Apr. 20, 1987 | Nov. 22, 1988 |
| 4,853,230 | Lovgren, et al. | Apr. 20, 1987 | Aug. 1, 1989 |
| 5,026,560 | Makino, et al. | Jan. 14, 1988 | Jun. 25, 1991 |
| 5,045,321 | Makino, et al. | Feb. 13, 1997 | Sep. 3, 1991 |
| 5,093,132 | Makino, et al. | Aug. 31, 1990 | Mar. 3, 1992 |
| 5,433,959 | Makino, et al. | Sep. 10, 1993 | Jul. 18, 1995 |
| 5,576,025 | Akiyama, et al. | Mar. 29, 1995 | Nov. 19, 1996 |
| 5,639,478 | Makino, et al. | Jun. 7, 1995 | Jun. 17, 1997 |
| 5,703,110 | Naka, et al. | Sep. 17, 1996 | Dec. 30, 1997 |
| 5,705,517 | Naka, et al. | Oct. 5, 1993 | Jan. 6, 1998 |
| 5,731,006 | Akiyama, et al. | Aug. 20, 1996 | Mar. 24, 1998 |
| 5,824,339 | Shimizu, et al. | Sep. 5, 1996 | Oct. 20, 1998 |
| 5,855,914 | Koyama, et al. | Aug. 9, 1994 | Jan. 5, 1999 |
| 5,879,708 | Makino, et al. | Feb. 27, 1997 | Mar. 9, 1999 |
| 5,948,773 | Akiyama, et al. | May 27, 1997 | Sep. 7, 1999 |
| 6,017,560 | Makino, et al. | Nov. 19, 1998 | Jan. 25, 2000 |
| 6,123,962 | Makino, et al. | Oct. 29, 1999 | Sep. 26, 2000 |
| 6,187,340 | Fakuta, et al. | Sep. 9, 1998 | Feb. 13, 2001 |
| 6,296,875 | Makino, et al. | Jun. 7, 2000 | Oct. 2, 2001 |
| 6,319,904 | Akiyama, et al. | Jul. 7, 1999 | Nov. 20, 2001 |
| 6,328,994 | Shimizu, et al. | May 17, 1999 | Dec. 11, 2001 |
| 4,255,431 | Junggren, et al. | Apr. 5, 1979 | Mar. 10, 1981 |
| 4,508,905 | Junggren, et al. | Apr. 6, 1983 | Apr. 2, 1985 |
| 4,636,499 | Brandstom, et al. | May 30, 1985 | Jan. 13, 1987 |
| 4,738,974 | Brandstrom | Apr. 21, 1986 | Apr. 19, 1988 |
| 5,690,960 | Bengtsson, et al. | Sep. 27, 1994 | Nov. 25, 1997 |
| 5,714,504 | Lindberg, et al. | Jan. 23, 1995 | Feb. 3, 1998 |
| 5,753,265 | Bergstrand, et al. | Jun. 22, 1995 | May 19, 1998 |
| 5,817,338 | Bergstrand, et al. | Jun. 20, 1995 | Oct. 6, 1998 |
| 6,013,281 | Depui, et al. | Mar. 8, 1996 | Jan. 11, 2000 |
| 6,136,344 | Depui, et al. | Apr. 15, 1996 | Oct. 24, 2000 |
| 6,159,499 | Seth | Oct. 20, 1998 | Dec. 12, 2000 |
| 6,183,776 | Depui, et al. | Feb. 13, 1997 | Feb. 6, 2001 |
| 6,328,994 | Shimizu, et al. | Aug. 4, 1999 | Dec. 11, 2001 |
| 6,479,075 | Odidi, et al. | Jan. 22, 2001 | Nov. 12, 2002 |
| 6,602,522 | Chen, et al. | Jun. 20, 2000 | Aug. 5, 2003 |

When a dosage form using the typical composition, thickness, amount, and make up of the enteric coating described in the above patents is administered to a subject, the majority of the pharmaceutical agent present in the dosage form is generally released in the lower gastrointestinal tract, thus there is little or no drug delivery at the desired location in the stomach and/or at a predetermined time after administration. However, these enteric coated dosage forms can in one embodiment meet the desired dosage release profile of the present invention when provided in conjunction with a buffering agent as described herein. In this embodiment, the buffering agent raises pH of gastrointestinal fluid to a pH for a period of time that substantially dissolves or disperses the enteric coating thereby releasing the pharmaceutical agent from the enteric coated dosage form into gastrointestinal fluid. The buffering agent by raising the pH to a level that dissolves or disperses the enteric coating, also acts to substantially prevent or protect the acid-labile pharmaceutical agent from acid degradation by reducing acidic conditions in the gastrointestinal fluid.

As used in the present invention, the term “disintegrate” includes the dissolving of an enteric coating in gastrointestinal fluid, and the subsequent dissolution and dispersion of the dosage form into the gastrointestinal fluid. The term “disintegrate” also refers to the enteric coating's loss of integrity as a barrier to gastrointestinal fluid, and to its loss of functionality as a gastrointestinal fluid protectant. The components of the coating generally disintegrates in gastrointestinal fluid within about 2 minutes or less, or within about 90 minutes or less, but these amounts may vary depending on the application and desired therapeutic effect as described herein.

The release of the proton pump inhibiting agent or other acid labile drug from the inventive compositions of the present invention can be determined by in vitro methods such as those described in U.S. Pharmacopoeia (USP 26-NF 1), which is herein incorporated by reference, including, for example, USP <724>, Drug Release; and USP <711>, Dissolution, or other standard in vitro dissolution assay techniques known in the art. Illustratively, formulations of the present invention can be tested for in vitro dissolution and/or disintegration properties using USP Dissolution Apparatus 2 at a paddle speed of 50 rpm. The formulations can be dissoluted using a one-, two-, or multiple-stage dissolution medium equilibrated to 37° C. In the two-and multiple-stage dissolution media, the dissolution media can be adjusted to various pH points to determine dissolution profiles over a range of pHs. For analysis of pH, an Orion pH Meter (model 720A) equipped with an Orion pH electrode (combination probe/PerpHeot Ross Semimicro Electrode) can be employed, for example. For simulated gastrointestinal fluid, 0.1N hydrochloric acid, with or without pepsin (pH less than 6.8) or pancreatin (pH greater than or equal to 6.8); pH 6.8 phosphate buffer prepared by mixing 0.1 N hydrochloric acid and 0.2 M tribasic sodium phosphate (3: 1), and adjusting the pH, if necessary; and/or simulated gastrointestinal fluid, USP 26-NF 21; can be utilized, for example. The pH of the dissolution media can be adjusted to a desired pH by using, for example, 0.2 M tribasic sodium phosphate, 2 N hydrochloric acid, and/or 2 N sodium hydroxide. Aliquots from the dissolution media can be taken over time, and the amount of acid-labile drug released into the dissolution media can be determined by, for example, using High Performance Liquid Chromatography (HPLC). A dissolution, disintegration and/or release profile over time and at various pH points can then be calculated. An enteric coating of the present invention can be tested with or without buffering agent to determine the dissolution and/or disintegration profile of the particular enteric coating (for example, thickness of various enteric coatings) at various pH points. The effect on the pH of the simulated gastrointestinal fluid of a buffering agent that may be present in a composition during testing can be determined by, for example, the Kinetic Acid Neutralization Model, described below.

Briefly, in the Kinetic Acid Neutralization Model the timed acid neutralization of an amount of buffering agent or agents (for example, a representative amount of calcium carbonate, and/or sodium bicarbonate, and/or sodium carbonate) can be evaluated. While not wishing to be bound by theory, it is generally believed that a healthy human stomach adds hydrochloric acid to the stomach contents at the rate of 30 ml per hour. The Kinetic Acid Neutralization Model uses a glass flask (in the form of a 100 ml or 200 ml dissolution flask, for example) to hold 0.1 N hydrochloric acid (HCl) (to simulate the acidity of the stomach in the fasted state). Fifty ml is considered the volume of gastrointestinal fluid usually found in a fasted stomach, and for experimental convenience, the model can, for example, utilize 100 ml (double the usual fasted stomach volume) of fluid and a corresponding doubling of the amount of buffering agent and/or acid-labile pharmaceutical agent tested. An overhead stirrer maintains at a constant, controlled and reproducible rpm, stirring the contents in the flask. For the analysis of pH, any type of pH monitor can be utilized, including for example, an Orion pH Meter (model 720A) equipped with an Orion pH electrode (for example, a combination probe/PerpHeot Ross Semimicro Electrode). The Kinetic Acid Neutralization Model can add by a peristaltic pump (for example, a Watson/Marlow Multichannel PumpPro model with acid resistant tubing), 200 ml per hour of 0.05 N hydrochloric acid. This rate compensates for the doubling of the initial volume of 0.1 N hydrochloric acid from 50 to 100 ml. To simulate stomach emptying, fluid can be withdrawn from the flask at the same rate and by the same peristaltic pump, maintaining the 100 ml volume constant. This Kinetic Acid Neutralization Model combines the concepts of USP (U.S. Pharmacopoeia) <301>, Acid-Neutralizing Capacity Test, and the concepts of USP <724>, the Flow Through Cell for Drug Release Testing, which are incorporated herein by reference. Illustratively, the pH of the initial acid in the flask can be measured as a function of time. At time zero, the buffering agent is added to the flask, and the pH of the contents measured, starting at one minute intervals, and progressing at convenient time intervals until the pH falls below a predetermined level, for example, a value of 3 or less. When testing a controlled-release dosage form of the present invention in this model, the amount of the acid-labile pharmaceutical agent released from the dosage form into the gastrointestinal fluid and/or the acid-degradation of the agent can be determined by, for example, using High Performance Liquid Chromatography (HPLC), or other standard assay techniques known in the art.

The acid resistance of a controlled-release layer (for example, an enteric coating) of the present invention can also be determined by exposing a dosage form to simulated gastrointestinal fluid, USP 26-NF 21, or 0.1 M hydrochloric acid (aqueous). Acid resistance is generally defined as the amount of active substance in a dosage form after being exposed to such fluid relative to that of unexposed dosage form, respectively. The test can, for example, be accomplished in the following way. A dosage form, for example, a tablet or pellet, is exposed to simulated gastrointestinal fluid at a temperature of 37° C. The dosage form disintegrates and releases the enteric coated acid labile drug into the medium. After a predetermined time the enteric coated layered drug is removed and analyzed for acid-degraded and/or non-acid-degraded drug content using High Performance Liquid Chromatography (HPLC). Acid resistance at various pH points, as well as disintegration or release profiles over time can then be calculated.

In one embodiment of the present invention, the enteric coating of a composition is a thickness that provides for release of at least 85% of the proton pump inhibiting agent in vitro within about 90 minutes using one or more of the in vitro tests described above. In other embodiments, the enteric coating of a composition is a thickness that provides for release of at least about 80% of the proton pump inhibiting agent in vitro within about 90 minutes. In another embodiment of the present invention, the enteric coating of a composition is a thickness that provides for release of at least about 75% of the proton pump inhibiting agent in vitro within about 90 minutes. In still another embodiment, the enteric coating of a composition is a thickness that provides for release of at least about 70% of the proton pump inhibiting agent in vitro within about 90 minutes. In yet another embodiment, the enteric coating of a composition is a thickness that provides for release of at least about 60% of the proton pump inhibiting agent in vitro within about 90 minutes. And n still another embodiment, the enteric coating of a composition is a thickness that provides for release of at least about 50% of the proton pump inhibiting agent in vitro within about 90 minutes.

In one embodiment of the present invention, the enteric coating of a composition is a thickness that provides for release of at least 85% of the proton pump inhibiting agent in vitro within about 60 minutes using one or more of the in vitro tests described above. In other embodiments, the enteric coating of a composition is a thickness that provides for release of at least about 80% of the proton pump inhibiting agent in vitro within about 60 minutes. In another embodiment of the present invention, the enteric coating of a composition is a thickness that provides for release of at least about 75% of the proton pump inhibiting agent in vitro within about 60 minutes. In still another embodiment, the enteric coating of a composition is a thickness that provides for release of at least about 70% of the proton pump inhibiting agent in vitro within about 60 minutes. In yet another embodiment, the enteric coating of a composition is a thickness that provides for release of at least about 60% of the proton pump inhibiting agent in vitro within about 60 minutes. And n still another embodiment, the enteric coating of a composition is a thickness that provides for release of at least about 50% of the proton pump inhibiting agent in vitro within about 60 minutes.

In one embodiment of the present invention, the enteric coating of a composition is a thickness that provides for release of at least 85% of the proton pump inhibiting agent in vitro within about 45 minutes using one or more of the in vitro tests described above. In other embodiments, the enteric coating of a composition is a thickness that provides for release of at least about 80% of the proton pump inhibiting agent in vitro within about 45 minutes. In another embodiment of the present invention, the enteric coating of a composition is a thickness that provides for release of at least about 75% of the proton pump inhibiting agent in vitro within about 45 minutes. In still another embodiment, the enteric coating of a composition is a thickness that provides for release of at least about 70% of the proton pump inhibiting agent in vitro within about 45 minutes. In yet another embodiment, the enteric coating of a composition is a thickness that provides for release of at least about 60% of the proton pump inhibiting agent in vitro within about 45 minutes. And n still another embodiment, the enteric coating of a composition is a thickness that provides for release of at least about 50% of the proton pump inhibiting agent in vitro within about 45 minutes.

In one embodiment of the present invention, the enteric coating of a composition is a thickness that provides for release of at least 85% of the proton pump inhibiting agent in vitro within about 30 minutes using one or more of the in vitro tests described above. In other embodiments, the enteric coating of a composition is a thickness that provides for release of at least about 80% of the proton pump inhibiting agent in vitro within about 30 minutes. In another embodiment of the present invention, the enteric coating of a composition is a thickness that provides for release of at least about 75% of the proton pump inhibiting agent in vitro within about 30 minutes. In still another embodiment, the enteric coating of a composition is a thickness that provides for release of at least about 70% of the proton pump inhibiting agent in vitro within about 30 minutes. In yet another embodiment, the enteric coating of a composition is a thickness that provides for release of at least about 60% of the proton pump inhibiting agent in vitro within about 30 minutes. And n still another embodiment, the enteric coating of a composition is a thickness that provides for release of at least about 50% of the proton pump inhibiting agent in vitro within about 30 minutes.

In one embodiment of the present invention, the enteric coating of a composition is a thickness that provides for release of at least 85% of the proton pump inhibiting agent in vitro within about 15 minutes using one or more of the in vitro tests described above. In other embodiments, the enteric coating of a composition is a thickness that provides for release of at least about 80% of the proton pump inhibiting agent in vitro within about 15 minutes. In another embodiment of the present invention, the enteric coating of a composition is a thickness that provides for release of at least about 75% of the proton pump inhibiting agent in vitro within about 15 minutes. In still another embodiment, the enteric coating of a composition is a thickness that provides for release of at least about 70% of the proton pump inhibiting agent in vitro within about 15 minutes. In yet another embodiment, the enteric coating of a composition is a thickness that provides for release of at least about 60% of the proton pump inhibiting agent in vitro within about 15 minutes. And n still another embodiment, the enteric coating of a composition is a thickness that provides for release of at least about 50% of the proton pump inhibiting agent in vitro within about 15 minutes.

In one embodiment of the present invention, the compositions prepared in accordance with the present invention provides a release profile of the acid-labile pharmaceutical agent into gastrointestinal fluid that results in about 50% or more of the acid-labile pharmaceutical agent released from the composition within about 10 to about 90 minutes, or within less than about 10, 11, 12, 13, 14, 15, 16, 17, 18, 19, 20, 30, 40, 50, 60, 70, 80, or 90 minutes; or at least about 80% or more of the acid-labile pharmaceutical agent released within about 10 to about 90 minutes, or within less than about 10, 11, 12, 13, 14, 15, 16, 17, 18, 19, 20, 30, 40, 50, 60, 70, 80, or 90 minutes; or at least about 90% or more of the acid-labile pharmaceutical agent released within about 10 to about 90 minutes, or within less than about 10, 11, 12, 13, 14, 15, 16, 17, 18, 19, 20, 30, 40, 50, 60, 70, 80, or 90 minutes; or at least about 95% or more of the acid-labile pharmaceutical agent released within about 10 to about 90 minutes, or within less than about 10, 11, 12, 13, 14, 15, 16, 17, 18, 19, 20, 30, 40, 50, 60, 70, 80, or 90 minutes; or at least about 99% or more of the acid-labile pharmaceutical agent released within about 10 to about 90 minutes, or within less than about 10, 11, 12, 13, 14, 15, 16, 17, 18, 19, 20, 30, 40, 50, 60, 70, 80, or 90 minutes; after exposure to the gastrointestinal fluid.

In another embodiment of the present invention, the compositions prepared in accordance with the present invention provides a release profile of the acid-labile pharmaceutical agent into gastrointestinal fluid that results in about 50% to about 85% of the acid-labile pharmaceutical agent being released from the composition within about 20 minutes, or not less than about 85%, or not less than about 80%, or not less than about 75%, or not less than about 50%, or not less than about 25%, or not less than about 10%, of the acid-labile pharmaceutical agent being released into the gastrointestinal fluid within about 20 minutes after exposure to the gastrointestinal fluid.

In another embodiment of the present invention, the compositions prepared in accordance with the present invention provides a release profile of the acid-labile pharmaceutical agent into gastrointestinal fluid that results in about 50% to about 85% of the acid-labile pharmaceutical agent being released from the composition within about 30 minutes, or not less than about 85%, or not less than about 80%, or not less than about 75%, or not less than about 50%, or not less than about 25%, or not less than about 10%, of the acid-labile pharmaceutical agent being released into the gastrointestinal fluid within about 30 minutes after exposure to the gastrointestinal fluid.

In another embodiment of the present invention, the compositions prepared in accordance with the present invention provides a release profile of the acid-labile pharmaceutical agent into gastrointestinal fluid that results in about 50% to about 85% of the acid-labile pharmaceutical agent being released from the composition within about 45 minutes, or not less than about 85%, or not less than about 80%, or not less than about 75%, or not less than about 50%, or not less than about 25%, or not less than about 10%, of the acid-labile pharmaceutical agent being released into the gastrointestinal fluid within about 45 minutes after exposure to the gastrointestinal fluid.

In another embodiment of the present invention, the compositions prepared in accordance with the present invention provides a release profile of the acid-labile pharmaceutical agent into gastrointestinal fluid that results in about 50% to about 85% of the acid-labile pharmaceutical agent being released from the composition within about 60 minutes, or not less than about 85%, or not less than about 80%, or not less than about 75%, or not less than about 50%, or not less than about 25%, or not less than about 10%, of the acid-labile pharmaceutical agent being released into the gastrointestinal fluid within about 60 minutes after exposure to the gastrointestinal fluid.

In another embodiment of the present invention, the compositions prepared in accordance with the present invention provides a release profile of the acid-labile pharmaceutical agent into gastrointestinal fluid that results in about 50% to about 85% of the acid-labile pharmaceutical agent being released from the composition within about 90 minutes, or not less than about 85%, or not less than about 80%, or not less than about 75%, or not less than about 50%, or not less than about 25%, or not less than about 10%, of the acid-labile pharmaceutical agent being released into the gastrointestinal fluid within about 90 minutes after exposure to the gastrointestinal fluid.

In yet another embodiment of the present invention, the composition is formulated to provide a composition that contains an enteric coated acid-labile pharmaceutical agent, for example, a proton pump inhibitor, and prior to administration to a subject, the enteric coating is substantially dissolved or removed from the acid-labile pharmaceutical agent when the composition is mixed with water or other aqueous media to produce a solution or suspension.

In one embodiment of the present invention, ingredients and carrier materials for a composition are selected to provide a disintegration or release profile of the enteric coating of the present invention in gastrointestinal fluid within about 10 minutes to about 90 minutes, or about 20 minutes to about 90 minutes, or about 30 minutes to about 45 minutes, or about 20-minutes to about 45 minutes, or less than about 10, 11, 12, 13, 14, 15, 16, 17, 18, 19, 20, 30, 40, 50, 60, 70, 80, or 90 minutes, releasing substantially all of the acid-labile pharmaceutical agent from the controlled-release component into the gastrointestinal fluid.

In another embodiment of the present invention, ingredients and carrier materials for a composition are selected to provide a disintegration profile of the enteric coating from which about 50% or more of the acid-labile pharmaceutical agent is released from the composition in vitro within about 10 minutes to about 90 minutes, or less than about 10, 11, 12, 13, 14, 15, 16, 17, 18, 19, 20, 30, 40, 50, 60, 70, 80, or 90 minutes; or at least about 80% or more of the acid-labile pharmaceutical agent is released in vitro within about 10 minutes to about 90 minutes, or less than about 10, 11, 12, 13, 14, 15, 16, 17, 18, 19, 20, 30, 40, 50, 60, 70, 80, or 90 minutes; or at least about 85% or more of the acid-labile pharmaceutical agent is released in vitro within about 10 minutes to about 90 minutes, or less than about 10, 11, 12, 13, 14, 15, 16, 17, 18, 19, 20, 30, 40, 50, 60, 70, 80, or 90 minutes; using 0.1 N hydrochloric acid in water at 37° C. in the dissolution assays discussed herein.

In one aspect, the present invention is directed to a composition that provides for release of at least one proton pump inhibiting agent from the controlled-release component that is coated with an enteric coating into gastrointestinal fluid where the enteric coating has a defined thickness. Typically, application of a thicker coating (greater than 20 μm, for example) will increase the time for complete release at any given pH level. Accordingly, in one aspect of the present invention, the release of the acid-labile pharmaceutical agent is accomplished by employing a predetermined thickness of an enteric coating that substantially dissolves between a pH of about 3 to about 8, within less than about 120 minutes, for example, less than about 1, 2, 3, 4, 5, 6, 7, 8, 9, 10, 11, 12, 13, 14, 15, 16, 17, 18, 19, 20, 30, 40, 50, 60, 70, 80, 90, 100, or 110 minutes.

In one embodiment of the present invention, the average thickness of the enteric coating is about 100 microns or less, or about 50 microns or less, or about 0.001 microns to about 20 microns, or about 0.05 microns to about 15 microns, or about 0.1 micron to about 10 microns. Illustratively, the average thickness of the enteric coating is about 100 microns, or about 90 microns, or about 80 microns, or about 70 microns, or about 60 microns, or about 50 microns, or about 40 microns, or about 30 microns, or about 20 microns, or about 15 microns, or about 10 microns, or about 5 microns, or about 2 microns, or about 1 micron, or about 0.5 microns, or about 0.25 microns, or about 0.1 microns, or about 0.05 microns, or about 0.01 microns.

In yet another embodiment of the present invention, the enteric coating of the controlled-release component has an average thickness of about 0.001 microns to about 100 microns, or about 0.01 microns to about 50 microns. Illustratively, the enteric coating has a thickness less than about 25 microns, or less than about 20 microns, or less than about 15 microns, or less than about 10 microns.

In yet another embodiment, a composition of the present invention contains a controlled-release component having an enteric coating of a thickness that provides for release of at least 75% of the acid-labile pharmaceutical agent from the composition in vitro within about 60 minutes in about 50 ml of 0.1N hydrochloric acid in water at 37° C.

In yet another embodiment a composition of the present invention contains a controlled-release component having an enteric coating of a thickness that provides for release of at least 75% of the acid-labile pharmaceutical agent from the composition in vitro within about 60 minutes in about 50 ml 1% sodium dodecyl sulfate in water at 37° C.

The following is an exemplary list of ingredients that can make up an enteric coating of the controlled-release layer of the present invention that results in the desired release profile: Acetylatedmonoglyceride; Carboxymethyl cellulose; Cellulose acetate phthalate; Cetyl alcohol; Citric acid anhydrous; Colorant; Diethyl phthalate; Eudragit® L-30D-55; Eudragit® NE30D; Eudragit® L 100; Eudragit® L 100-55; Eudragit® S100; Eudragit® FS 30 D; Glyceryl monostearate; Hydrogen peroxide; Hydroxypropylmethylcellulose phthalate; Hydroxypropylmethylcellulose; Hydroxypropyl methylcellulose acetate succinate; KollICoat MAE30DP; Macrogel 6000; Methacrylic acid copolymer; Mono-and diglycerides; Polyethylene glycol 6000; Polyethylene glycol; Polyethylene glycol 400; Polyethylene glycol 6000; Polyquid PA-30; Polysobate 80; Shellac; Sodium laurylsulphate; Stabalizer; Stearyl alcohol; Talc; Triacetin; Triethyl citrate; and Tween® 80.

Discussion of other enteric coating materials useful in the present invention are also discussed in, for example, Remington: The Science and Practice of Pharmacy,Nineteenth Ed. (Easton, Pa.: Mack Publishing Company, 1995). Another discussion on enteric coating materials can be found in Hoover, John E., Remington's Pharmaceutical Sciences, Mack Publishing Co., Easton, Pa. 1975. Another discussion on enteric coating materials can be found in Liberman, H. A. and Lachman, L., Eds., Pharmaceutical Dosage Forms, Marcel Decker, New York, N.Y., 1980. Another discussion on enteric coating materials can be found in Pharmaceutical Dosage Foms and Drug Delivery Systems, Seventh Ed. (Lippincott Williams & Wilkins 1999). Illustratively, respective compositions of enteric coatings that can be used in the present invention are provided below in Table Nos. 2-14. In one embodiment of the present invention, enteric coated granules are generally produced by coating a granule produced by methods known to those skilled in the art (see Table No. 1) with an enteric coating composition specified in Table Nos. 2-14 below. For example, the enteric coated granules can be produced using a fluidized bed granulator (Okawara, Japan) under conditions such that inlet air temperature is about 50° C. and the granule temperature is about 40° C. In one embodiment of the present invention, the ingredients of the illustrative enteric coated compositions of Table Nos. 3-13, are thoroughly mixed together to obtain dusting powders for application to the granule or particle.

TABLE NO. 3
Illustrative Enteric Coating Compositions

| Ingredient | Ex. 1 | Ex. 2 | Ex. 3 | Ex. 4 | Ex. 5 | Ex. 6 |
|---|---|---|---|---|---|---|
| Eudragit ® | 138 mg | 628 g | 628 g | | 2018 g | |
| L-30D-55 | | | | | | |
| Talc | 4.1 mg | 192 g | 192 g | 20 g | 1832 g | |
| Polyethylene | 12.4 mg | 192 g | 64 g | | 60 g | |
| glycol 6000 | | | | | | |
| Hydroxy- | | | | 1000 g | | 11.6 kg |
| propyl- | | | | | | |
| methyl- | | | | | | |
| cellulose | | | | | | |
| phthalate | | | | | | |
| 220824 | | | | | | |
| Tween ® 80 | 2.1 mg | 64 g | 32 g | | 27 g | |
| Ethanol | | | | | | 56.3 kg |
| Castor oil | | | | 100 g | | |
| Acetone | | | | 101 | | 131.5 kg |
| Shellac | | | | | | 2.8 kg |
| Colorant | | 64 g | 64 g | | 60 g | |
| Water | 276 μl | 4.4 l | 4.4 l | | 4.23 l | |

TABLE NO. 4
Illustrative Enteric Coating Compositions

| Ingredient | Ex. 7 | Ex. 8 | Ex. 9 | Ex. 10 | Ex. 11 | Ex. 12 |
|---|---|---|---|---|---|---|
| Eudragit ® | 1789 g | | 1078 g | 5016 g | 1911 g | 2290 g |
| L-30D-55 | | | | | | |
| Eudragit ® | | | 138.5 g | 559 g | 212.9 g | 253 g |
| NE30D | | | | | | |
| Talc | 161 g | | 16 g | | | 53 g |
| Polyethylene | | 0.6 mg | | | | |
| glycol 6000 | | | | | | |
| Macrogel | 52.8 g | | | | | |
| 6000 | | | | | | |
| Hydroxy- | | 2.592 mg | | | | |
| propyl- | | | | | | |
| methyl- | | | | | | |
| cellulose | | | | | | |
| 2910 | | | | | | |
| Triethyl | | | 46 g | 333.7 g | 127.1 g | 153 g |
| citrate | | | | | | |
| Glyceryl | | | 23.1 g | 106.5 g | 40.6 g | 20 g |
| monostearate | | | | | | |
| Polysorbate | 24 g | | 9 g | 34.8 g | 13.3 g | 8 g |
| 80 | | | | | | |
| Stabalizer | | 0.4 mg | | | | |
| Colorant | 52.8 g | 0.408 mg | 0.5 g | 1.8 g | 0.8 g | 53 g |
| Water | 3744 g | | 2039 g | 2547 g | 970.3 g | 2420 g |

TABLE NO. 5
Illustrative Enteric Coating Compositions

| Ingredient | Ex. 13 | Ex. 14 | Ex. 15 | Ex. 16 | Ex. 17 | Ex. 18 |
|---|---|---|---|---|---|---|
| Eudragit ® L- | 1219 g | 4032 g | | 610.4 g | 1017 g | 1078 g |
| 30D-55 | | | | | | |
| Eudragit ® | 134.4 g | 447.8 g | | 68 g | 373.2 g | 138.5 g |
| NE30D | | | | | | |
| Talc | | | | | | 16 g |
| Polyethylene | 40.8 g | | 20.4 g | 20.4 g | | |
| glycol 6000 | | | | | | |
| Triethyl citrate | | 269.3 g | | | 224.4 g | 46 g |
| Glyceryl | 24 g | 86.4 g | 12 g | 12 g | 72 g | 16.5 g |
| monostearate | | | | | | |
| Citric acid | 0.48 g | 0.72 g | 0.24 g | 0.24 g | 0.6 g | |
| anhydrous | | | | | | |
| Polysorbate 80 | 7.2 g | 25.9 g | 3.6 g | 3.6 g | 21.6 g | 9 g |
| Colorant | 0.48 g | 1.72 g | 0.24 g | 0.24 g | 1.44 g | 0.5 g |
| Water | 1693 g | 2624 g | 846.7 g | 845.1 g | 1707 g | 2039 g |

TABLE NO. 6
Illustrative Enteric Coating Compositions

| Ingredient | Ex. 1 | Ex. 2 | Ex. 3 | Ex. 4 | Ex. 5 | Ex. 6 |
|---|---|---|---|---|---|---|
| Methacrylic | 100 g | 9000 g | 200 g | 124 g | 250 g | |
| acid | | | | | | |
| copolymer | | | | | | |
| Hydroxypropyl | | | | | | 400 g |
| methylcellulose | | | | | | |
| phthalate | | | | | | |
| Triethyl | 30 g | 2700 g | 60 g | | | |
| citrate | | | | | | |
| Mono- and | 5 g | 450 g | | 3 g | 12.5 g | |
| diglycerides | | | | | | |
| Polysorbate | 0.5 g | 40 g | | 1 g | 1.2 g | |
| 80 | | | | | | |
| Polyethylene | | | | 25 g | | |
| glycol 400 | | | | | | |
| Polyethylene | | | | | 75 g | |
| glycol 6000 | | | | | | |
| Diethyl | | | | | | 80 g |
| phthalate | | | | | | |
| Ethanol | | | | | | 1600 g |
| Acetone | | | | | | 4000 g |
| Purified water | 309 g | 19000 g | 392 g | 463 g | 490 g | |

TABLE NO. 7
Illustrative Enteric Coating Compositions

| Ingredient | Ex. 7 | Ex. 8 | Ex. 9 | Ex. 10 | Ex. 11 | Ex. 12 |
|---|---|---|---|---|---|---|
| Methacrylic acid | 400 g | 140 g | 500 g | 256 g | | 200 g |
| copolymer | | | | | | |
| Hydroxypropyl | | | | | 100 g | |
| methylcellulose | | | | | | |
| acetate succinate | | | | | | |
| Triethyl citrate | 120 g | 42 g | 150 g | | 30 g | 60 g |
| Mono- and | | 7 g | 25 g | | | 10 g |
| diglycerides | | | | | | |
| Polysorbate 80 | | 0.7 g | 2.5 g | | | 1 g |
| Polyethylene | | | | 64 g | | |
| glycol 400 | | | | | | |
| Ethanol | | | | | 720 g | |
| Talc | 120 g | | | | | |
| Purified water | | 300 g | 978 g | 1217 g | 309 g | 391 g |

TABLE NO. 8
Illustrative Enteric Coating Compositions

| Ingredient | Ex. 13 | Ex. 14 | Ex. 15 | Ex. 16 | Ex. 17 | Ex. 18 |
|---|---|---|---|---|---|---|
| Methacrylic | 40 g | 120 g | | 9.1 mg | 9.1 mg | 9.1 mg |
| acid | | | | | | |
| copolymer | | | | | | |
| Cellulose | | | 375 g | | | |
| acetate | | | | | | |
| phthalate | | | | | | |
| Triethyl citrate | 12 g | 36 g | | | | |
| Mono- and | 2 g | 6 g | | | | |
| diglycerides | | | | | | |
| Polysorbate 80 | 0.2 g | 0.6 g | | | | |
| Polyethylene | | | | 1 mg | 1 mg | 1 mg |
| glycol | | | | | | |
| Diethyl | | | 150 g | | | |
| phthalate | | | | | | |
| Ethanol | | | 2000 g | | | |
| Acetone | | | 2000 g | | | |
| Colorant | | | | 0.06 mg | 0.13 mg | 0.43 mg |
| Purified water | 78 g | 235 g | | 45 mg | 45 mg | 45 mg |

TABLE NO. 9
Illustrative Enteric Coating Compositions

| Ingredient | Ex. 19 | Ex. 20 | Ex. 21 | Ex. 22 | Ex. 23 | Ex. 24 |
|---|---|---|---|---|---|---|
| Eudragit ® | | | | | | 45 g |
| L 100 | | | | | | |
| Hydroxypropyl | | 70 g | 57 g | 500 g | 45 g | |
| methylcellulose | | | | | | |
| phthalate | | | | | | |
| Cellulose | 200 g | | | | | |
| acetate | | | | | | |
| phthalate | | | | | | |
| Ethanol | | 600 g | 231 g | 219 g | 680 g | 1320 g |
| Cetyl alcohol | 15 g | 4 g | 3 g | 45 g | 5 g | |
| Stearyl alcohol | | | | | | 4.5 g |
| Isopropanol | 2000 g | | | | | |
| Methylene | 2000 g | | | | | |
| Chloride | | | | | | |
| Acetone | | 200 g | 540 g | 5 g | 371 g | |

TABLE NO. 10
Illustrative Enteric Coating Compositions

| Ingredient | Ex. 25 | Ex. 26 | Ex. 27 | Ex. 28 | Ex. 29 | Ex. 30 |
|---|---|---|---|---|---|---|
| Methacrylic | | | 200 g | 250 g | | 400 g |
| acid | | | | | | |
| copolymer | | | | | | |
| Hydroxypropyl | 1500 g | 7 mg | | | 400 g | |
| methylcellulose | | | | | | |
| phthalate | | | | | | |
| Triethyl citrate | | | 60 g | | | 120 g |
| Mono- and | | | 10 g | 10 g | | 8 g |
| diglycerides | | | | | | |
| Polysorbate 80 | | | 1 g | 1 g | | 1 g |
| Polyethylene | | | | 50 g | | |
| glycol 400 | | | | | | |
| Diethyl | | | | | 80 g | |
| phthalate | | | | | | |
| Ethanol | | | | | 1600 g | |
| Cetyl alcohol | 105 g | 0.5 mg | | | | |
| Isopropanol | 15000 g | | | | | |
| Methylene | 15000 g | | | | | |
| Chloride | | | | | | |
| Acetone | | | | | 4000 g | |
| Purified water | 3150 g | | 420 g | 650 g | | 800 g |

TABLE NO. 11
Illustrative Enteric Coating Compositions

| Ingredient | Ex. 31 | Ex. 32 | Ex. 33 | Ex. 34 | Ex. 35 | Ex. 36 |
|---|---|---|---|---|---|---|
| Eudragit ® | | | | 151.5 g | | |
| L30 D-55 | | | | | | |
| Methacrylic | 100 g | 50 g | | | | |
| acid | | | | | | |
| copolymer | | | | | | |
| Hydroxypropyl | | | 6.3 g | | 14.4 g | 49.2 g |
| methylcellulose | | | | | | |
| acetate | | | | | | |
| succinate | | | | | | |
| Triethyl citrate | 30 g | 15 g | 1.3 g | | 2.9 g | 9.8 g |
| Mono- and | 5 g | | | | | |
| diglycerides | | | | | | |
| Polysorbate 80 | 0.5 g | | | | | |
| Polyethylene | | | | 4.6 g | | |
| glycol 400 | | | | | | |
| Sodium | | | 0.2 g | | 0.4 g | 1.5 g |
| laurylsulphate | | | | | | |
| Talc | | 15 g | 1.9 g | | 4.3 g | 14.8 g |
| Purified water | 282 g | 125 g | 80 g | 93.9 g | 183 g | 626.8 g |

TABLE NO. 12
Illustrative Enteric Coating Compositions

| Ingredient | Ex. 37 | Ex. 38 | Ex. 39 | Ex. 40 | Ex. 41 | Ex. 42 |
|---|---|---|---|---|---|---|
| Methacrylic | 19800 g | 32700 g | 333.7 g | 100 g | 200 g | 124 g |
| acid | | | | | | |
| copolymer | | | | | | |
| Triethyl citrate | 1790 g | 2940 g | 30 g | 30 g | 60 g | |
| Mono- and | 297 g | 490 g | 5 g | 5 g | | 3 g |
| diglycerides | | | | | | |
| Polysorbate 80 | 30 g | 49 g | 0.5 g | 0.5 g | | 1 g |
| Polyethylene | | | | | | 25 g |
| glycol 400 | | | | | | |
| Purified water | 11640 g | 19190 g | 196 g | 309 g | 392 g | 463 g |

TABLE NO. 13
Illustrative Enteric Coating Compositions

| Ingredient | Ex. 43 | Ex. 44 | Ex. 45 | Ex. 46 | Ex. 47 | Ex. 48 |
|---|---|---|---|---|---|---|
| Methacrylic | 250 g | | 140 g | 38700 g | | 30000 g |
| acid | | | | | | |
| copolymer | | | | | | |
| Hydroxypropyl | | | | | 250 g | |
| methylcellulose | | | | | | |
| acetate | | | | | | |
| succinate | | | | | | |
| Cellulose | | 250 g | | | | |
| acetate | | | | | | |
| phthalate | | | | | | |
| Triethyl citrate | | | 42 g | 3480 g | | 2700 g |
| Mono- and | 12.5 g | | 7 g | 580 g | | 490 g |
| diglycerides | | | | | | |
| Polysorbate 80 | 1.2 g | | 0.7 g | 60 g | | 50 g |
| Polyethylene | 75 g | | | | 62.5 g | |
| glycol 6000 | | | | | | |
| Ethanol | | 1000 g | | | | |
| Cetyl alcohol | | 50 g | | | | |
| Acetone | | 2500 g | | | | |
| Hydrogen | | | | | 0.75 g | |
| peroxide | | | | | | |
| Colorant | | | | | 62.5 g | |
| Purified water | 490 g | | | | | |

TABLE NO. 14
Illustrative Enteric Coating Compositions

| Ingredient | Ex. 49 | Ex. 50 | Ex. 51 | Ex. 52 | Ex. 53 |
|---|---|---|---|---|---|
| Methacrylic acid | 667 g | 2450 g | 40000 g | 322.5 g | |
| copolymer | | | | | |
| Hydroxypropyl | | | | | 250 g |
| methylcellulose | | | | | |
| phthalate | | | | | |
| Triethyl citrate | 60 g | | 3600 g | 96.8 g | |
| Mono- and | 10 g | | 600 g | 16.1 g | |
| diglycerides | | | | | |
| Polysorbate 80 | 1 g | | 60 g | 1.61 g | |
| Polyethylene | | 80 g | | | |
| glycol 400 | | | | | |
| Ethanol | | | | | 1000 g |
| Cetyl alcohol | | | | | 50 g |
| Colorant | | 100 g | | | |
| Purified water | 391 g | 1960 g | 24450 g | 631.4 g | |

In another embodiment of the present invention, a thinner application of an enteric coating as described in the art, for example, a thinner application of an enteric coating as taught in the patents of Table No. 2, and/or the enteric coating described in Table Nos. 3-13, allows or facilitates the release of substantially all of the acid-labile pharmaceutical drug from a composition at the appropriate time and/or in the desired predetermined area of the gastrointestinal tract, for example, in the stomach.

When used in conjunction with the buffering agents of the present invention, the acid degradation of an acid-labile pharmaceutical agent can be substantially prevented or inhibited to an extent to where a gastrointestinal-disorder-effective amount or dose of the pharmaceutical agent in a substantially non-acid degraded or reacted form is absorbed into the bloodstream. The thickness that provides the appropriate release profile of the present invention can be experimentally determined as above, or by using, for example, the Kinetic Acid Neutralization Model described herein in conjunction with HPLC. The thickness of enteric coating that provides for the release of the acid-labile pharmaceutical agent into gastrointestinal fluid depends on many factors, including, for example, the composition of the enteric coating, the pH of the gastrointestinal fluid, the amount of buffering agent used in the dosage form, and if administered to a subject, the pH of stomach secretions prior to administration, the age, weight, sex, fed state and health of the subject, and the time period at which the dosage form is administered, for example, prior to bedtime or prior to, during or after a meal.

Hardness of the controlled-release coating can be measured on a Schleuniger hardness tester and ranges in one embodiment of the present invention from about 0.1N to about 200N. The flexibility/hardness of enteric coating layers can also be characterized for instance as Vickers hardness measured with a Shimadzu hardness indentation tester type HMV 2000. Illustratively, an enteric coating layer applied to a pellet or granule of the present invention has a Vickers hardness value ranging from about 0.1 to about 10, or of less than about 10, of less than about 8, or less than about 6, or less than about 4, or less than about 2, or less than about 1, or less than about 0.1. In case the pellets are covered with an over-coating layer the Vickers hardness of the enteric coating layer is generally characterized before the over-coating layer is applied.

The enteric coatings can further comprise hydrophilic and/or hydrophobic polymers or film forming compounds that optionally contain further additives that help to control the erosion of the enteric coating in aqueous media and/or control the permeation of aqueous media through the enteric coat to the core of the preparation containing the active drug substance.

Other suitable enteric coating materials for use in the preparation of controlled release compositions include, but are not limited to, any pharmaceutically acceptable polymer such as ethyl cellulose, cellulose acetate butyrate, cellulose acetates, polymethacrylates containing quaternary ammonium groups or other pharmaceutically acceptable polymers, polyethylene glycol, hydroxypropyl cellulose, hydroxypropyl methylcellulose, polyvinylpyrrolidone, and polyvinyl alcohol; monomeric materials such as sugars including lactose, sucrose, fructose and mannitol; salts including sodium chloride, potassium chloride and derivatives; organic acids including furmaric acid, succinic acid, lactic acid and tartaric acid and mixtures thereof; and other enteric polymers including polyvinyl acetate phthalate, cellulose acetate phthalate, cellulose acetate trimellitate, shellac, zein, and polymethacrylates containing carboxyl groups. These polymers can be applied as solutions or latexes. Other barriers may be used such as waxes.

The layers and coatings of the present invention can also be plasticized according to the properties of the coating blend such as the glass transition temperature of the main component or mixture of components or the solvent used for applying the coating compositions. Suitable plasticizers can be added from about 0% to about 50% by weight of the coating composition. Such plasticizers include, for example, the group consisting of diethyl phthalate, citrate esters, polyethylene glycol, glycerol, acetylated glycerides, and castor oil.

Mixtures of the various controlled-release layers and coatings can also be used to give the desired release profile of the pharmaceutical agent from the composition of the present invention.

The compositions can also include a controlled-release component for release of the acid-labile pharmaceutical agent into the gastrointestinal fluids in association with an acid-labile pharmaceutical agent for release into the intestinal tract. The form of such a composition can include an amount of a proton pump inhibiting agent for release into the gastrointestinal fluid that is about 0.5% to about 99.5% of the total amount of proton pump inhibiting agent of the composition, with the proton pump inhibiting agent that is released into the intestinal tract containing the remainder of the proton pump inhibiting agent (about 0.5% to about 99.5%). As a result, the final composition provides an amount of proton pump inhibiting agent for release into the gastrointestinal fluid and an additional amount of proton pump inhibiting agent for release into the intestine after administration. This combination provides for rapid absorption of a proton pump inhibiting agent from the stomach into the blood serum that provides for substantially immediate availability of the proton pump inhibiting agent at the site of therapeutic action while providing delayed absorption (and thus delayed therapeutic action) of a proton pump inhibiting agent from the intestinal tract, to provide for immediate symptomatic relief and extended symptomatic relief.

The compositions of the present invention can also contain one or more flavoring agents, sweetening agents, and/or colorants.

“Flavoring agents” useful in the present invention for improving the taste of a composition include, for example, acacia syrup, acesulfame potassium, alitame, anise, apple, aspartame, banana, Bavarian cream, berry, black currant, butter, butter pecan, butterscotch, calcium citrate, camphor, caramel, cherry, cherry cream, chocolate, cinnamon, citrus, citrus punch, citrus cream, cocoa, coffee, cola, cool cherry, cool citrus, cyclamate, cylamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, MagnaSweet®, maltol, mannitol, maple, menthol, mint, mint cream, mixed berry, neohesperidine DC, neotame, nut, orange, peanut butter, pear, peppermint, peppermint cream, Prosweetg Powder, raspberry, root beer, rum, saccharin, safrole, sorbitol, spearmint, spearmint cream, strawberry, strawberry cream, stevia, sucralose, sucrose, Swiss cream, tagatose, tangerine, thaumatin, tutti fruitti, vanilla, walnut, watermelon, wild cherry, wintergreen, xylitol, or any combination of these flavoring ingredients, for example, anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof. Further, a flavoring agent can include a film coating to affect taste of the composition, such as those described in U.S. Pat. Nos. 4,851,226, 5,075,114, and 5,876,759. Also see, Remingon: The Science and Practice of Pharmacy, Nineteenth Ed. (Easton, Pa.: Mack Publishing Company, 1995). Another discussion can be found in Hoover, John E., Remington's Pharmaceutical Sciences, Mack Publishing Co., Easton, Pa. 1975. Another discussion can be found in Liberman, H. A. and Lachman, L., Eds., Pharmaceutical Dosage Forms, Marcel Decker, New York, N.Y., 1980. Another discussion can be found in Pharmaceutical Dosage Foms and Drug Delivery Systems, Seventh Ed. (Lippincott Williams & Wilkins 1999).

“Sweetening agents” that can be used in the present invention include, for example, acesulfame potassium (acesulfame K), alitame, aspartame, cyclamate, cylamate, dextrose, isomalt, MagnaSweet®, maltitol, mannitol, neohesperidine DC, neotame, Prosweet® Powder, saccharin, sorbitol, stevia, sucralose, sucrose, tagatose, thaumatin, xylitol, and the like.

The compositions of the present invention can also contain one or more coatings for color identification such as, for example, Opadry™ White YS-1-18027A (or another color).

In various embodiments of the present invention, the enteric coating can also contain one or more agents that facilitates erosion or diffusion of the coating to facilitate the disintegration of the composition resulting in the release of the components from the composition into gastrointestinal fluid. Erosion facilitators include, for example, a material that controls the erosion of the controlled-release coating in gastrointestinal fluid, and are generally known to those of ordinary skill in the art. Exemplary materials include, without limitation, hydrophilic polymers, electrolytes, proteins, peptides, and amino acids. A diffusion facilitator include, for example, a material that controls the diffusion of an aqueous fluid through the controlled-release coating, and are generally known to those of ordinary skill in the art. Exemplary materials include, without limitation, hydrophilic polymers, electrolytes, proteins, peptides, and amino acids. Combinations of the above erosion facilitator and diffusion facilitator material can also be used in the compositions of the present invention.

In one embodiment of the present invention, the dosage form comprises a composition with one or more acid-labile pharmaceutical agents; one or more controlled-release layers of an enteric coating with various thickness over the one or more acid-labile pharmaceutical agents; and one or more buffering agents. The combination of the one or more buffering agents and the enteric coating surprisingly provides the unique release profile of the acid-labile pharmaceutical agents at the desired time in the desired area of the gastrointestinal tract. In one aspect, the one or more acid-labile pharmaceutical agents can be provided within or as a part of a core, particle, or granule around which the enteric coating is applied, and which make up the controlled-released component of a dosage form of the present invention.

The carrier materials that can be employed in making the compositions of the present invention are any of those commonly used excipients in pharmaceutics and should be selected on the basis of compatibility with the acid-labile pharmaceutical agent and the release profile properties of the desired dosage form. Illustratively, suitable pharmaceutical excipients include:
(a) Binders that impart cohesive qualities to a powdered material including, for example, alginic acid and salts thereof; cellulose derivatives, for example, carboxymethylcellulose, methylcellulose, ethylcellulose, hydroxypropylmethylcellulose, hydroxyethylcellulose, hydroxypropylcellulose, ethylcellulose (for example, Ethocel®), and microcrystalline cellulose; microcrystalline dextrose; amylose; magnesium aluminum silicate; polysaccharide acids; bentonites;; gelatin; polyvinylpyrrolidone/vinyl acetate copolymer; crospovidone; povidone; polymethacrylates, for example, Eugradit® NE30D and RS30D;;; starch; pregelatinized starch; tragacanth; dextrin; a sugar, for example, sucrose, glucose, dextrose, molasses, and lactose; a natural or synthetic gum, for example, acacia, ghatti gum, mucilage of isapol husks, polyvinylpyrrolidone (for example, Polyvidone® CL, Polyvidone®, Kollidon® CL, Polyplasdone® XL, Polyplasdone® XL-10), and larch arabogalactan; Veegum®; polyethylene glycol; waxes; sorbitol; sodium alginate;; and the like.
(b) Suspending agents such as polyvinylpyrrolidone, for example, polyvinylpyrrolidone K12, polyvinylpyrrolidone K17, polyvinylpyrrolidone K25, or polyvinylpyrrolidone K30; polyethylene glycol, for example, the polyethylene glycol can have a molecular weight of about 300 to about 6000, or about 3350 to about 4000, or about 7000 to about 5400; polysorbate-80; sodium alginate; gums, such as, for example, gum tragacanth and gum acacia; xanthans, including xanthan gum; sugars; cellulosics, such as, for example, sodium carboxymethylcellulose, methylcellulose, hydroxy-propylmethylcellulose, hydroxyethylcellulose; polysorbate-80; sodium alginate; polyethoxylated sorbitan monolaurate; polyethoxylated sorbitan monolaurate; and the like.
(c) Disintegration agents that facilitate breakup or disintegration of a substance including a starch, for example, a natural starch (for example, corn starch or potato starch), pregelatinized starch (for example, National 1551, or Amijel®), or sodium starch glycolate (for example, Promogel®, or Explotab®); a cellulose, for example, a wood product, methylcrystalline cellulose (for example, Avicel®, Avicel® PH101, Avicel® PH1102, Avicel® PH105, Elcema® P100, Emcocel®, Vivacel®, Ming Tia®, and Solka-Floc®), methylcellulose, sodium carboxymethylcellulose, croscarmellose, or carboxymethylcellulose (for example, Primogel®, and Explotab®; a cross-linked cellulose, for example, cross-linked sodium carboxymethylcellulose (Ac-Di-Sol®), cross-linked carboxymethylcellulose, or cross-linked croscarmellose; a cross-linked starch, for example, sodium starch glycolate; a cross-linked polymer, for example, crospovidone; a cross-linked polyvinylpyrrolidone; a calcium; alginate, for example, alginic acid or a salt of alginic acid (for example, sodium alginate); a clay, for example, Veegum® HV (magnesium aluminum silicate); a gum, for example, agar, guar, locust bean, Karaya, pectin, or tragacanth; sodium starch glycolate; bentonite; a natural sponge; a surfactant; a resin, for example, a cation-exchange resin; citrus pulp; sodium lauryl sulfate; sodium lauryl sulfate in combination starch; and the like.
(d) Filling agents such as lactose; calcium carbonate; calcium phosphate; dibasic calcium phosphate; calcium sulfate; microcrystalline cellulose; cellulose powder; dextrose; dextrates; dextran; starches; pregelatinized starch; sucrose; xylitol; lactitol; mannitol; sorbitol; sodium chloride; polyethylene glycol; and the like.
(e) Surfactants such as sodium lauryl sulfate; sorbitan monooleate; polyoxyethylene sorbitan monooleate; polysorbates; polaxomers; bile salts; glyceryl monostearate; a copolymer of ethylene oxide and propylene oxide, for example, Pluronic® (BASF); and the like.
(f) Solubilizer such as citric acid; succinic acid; fumaric acid; malic acid; tartaric acid; maleic acid; glutaric acid; sodium bicarbonate; sodium carbonate and the like.
(g) Stabilizers such as any antioxidation agents; buffers; acids; and the like.
(h) Lubricants that prevent, reduce or inhibit adhesion or friction of materials including stearic acid; calcium hydroxide; talc; sodium stearyl fumerate; a hydrocarbon, for example, mineral oil, or hydrogenated vegetable oil (for example, hydrogenated soybean oil (Sterotex®)); higher fatty acids and their alkali-metal and alkaline earth metal salts, such as magnesium, aluminum, calcium and sodium stearates, stearic acid; stearic acid; glyceryl; magnesium; talc; waxes; Stearowet®;; boric acid; sodium benzoate; sodium acetate; sodium chloride; leucine; a polyethylene glycol or a methoxypolyethylene glycol, for example, Carbowax™ (for example, Carbowax™ 4000 or 6000); sodium oleate; glyceryl behapate; polyethylene glycol; magnesium or sodium lauryl sulfate; colloidal silica, for example, Syloid™; Carb-O-Sil®; a starch, for example, corn starch; silicone oil; a surfactant; and the like.
(i) Wetting agents such as oleic acid; glyceryl monostearate; sorbitan monooleate; sorbitan monolaurate; triethanolamine oleate; polyoxyethylene sorbitan monooleate; polyoxyethylene sorbitan monolaurate; sodium oleate; sodium lauryl sulfate; and the like.
(j) Diluents that increase bulk of the composition to facilitate compression including, for example, lactose; starch; mannitol; sorbitol; dextrose; microcrystalline cellulose (for example, Avicel®); dibasic calcium phosphate; dicalcium phosphate dihydrate; tricalcium phosphate; calcium phosphate; anhydrous lactose; spray-dried lactose; pregelatinzed starch; compressible sugar, for example, Di-Pac® (Amstar); mannitol; hydroxypropylmethylcellulose; sucrose-based diluents; confectioner's sugar; monobasic calcium sulfate monohydrate; calcium sulfate dihydrate; calcium lactate trihydrate; dextrates; inositol; hydrolyzed cereal solids; amylose; powdered cellulose; calcium carbonate; glycine; kaolin; mannitol; sodium chloride; inositol; bentonite; and the like.
(k) Anti-adherents or glidants that improve flow characteristics of a material including colloidal silicon dioxide, for example, Cab-o-sil® (Cabot); tribasic calcium phosphate; talc; corn starch; DL-leucine; sodium lauryl sulfate; magnesium, calcium, or sodium stearate; and the like.
(l) Pharmaceutically compatible carrier comprises acacia; gelatin; colloidal silicon dioxide; calcium glycerophosphate; calcium lactate; maltodextrin; glycerine; magnesium silicate; sodium caseinate; soy lecithin; sodium chloride; tricalcium phosphate; dipotassium phosphate; sodium stearoyl lactylate; carrageenan; monoglyceride; diglyceride; pregelatinized starch; and the like.

In various embodiments of the present invention, combinations of the above carrier materials can also be used. Additionally, drug formulations and carrier materials useful in the present invention are discussed in, for example, Remington: The Science and Practice of Pharmacy, Nineteenth Ed. (Easton, Pa.: Mack Publishing Company, 1995). Another discussion of drug formulations and carrier materials can be found in Hoover, John E., Remington's Pharmaceutical Sciences, Mack Publishing Co., Easton, Pennsylvania 1975. Another discussion of drug formulations and carrier materials can be found in Liberman, H. A. and Lachman, L., Eds., Pharmaceutical Dosage Forms, Marcel Decker, New York, N.Y., 1980. Another discussion of drug formulations and carrier materials can be found in Pharmaceutical Dosage Foms and Drug Delivery Systems, Seventh Ed. (Lippincott Williams & Wilkins 1999).

In one embodiment of the present invention, the composition contains at least one excipient, a pharmaceutically compatible carrier, a binder, a filling agent, a suspending agent, a flavoring agent, a sweetening agent, a disintegrant, a flow aid, a lubricant, an adjuvant, a colorant, a diluent, a solubilizer, a moistening agent, a stabilizer, a wetting agent, an anti-adherent, a glidant, a preservative, a parietal cell activator, an anti-foaming agent, an antioxidant, a chelating agent, an antifungal agent, an antibacterial agent, an isotonic agent, and combinations thereof.

In making the compositions of the present invention, the controlled-release component and/or a buffering agent can be mixed with a pharmaceutically acceptable excipient, diluted by the excipient or enclosed within such a carrier, which can be in the form of a capsule, sachet, paper or other container. Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup and methyl cellulose. The formulations can additionally include: lubricating agents, such as talc, magnesium stearate and mineral oil; wetting agents; emulsifying and suspending agents; preserving agents, such as methyl-and propylhydroxybenzoates; sweetening agents; and flavoring agents.

When the excipient serves as a diluent, it can be a solid, semi-solid or liquid material, which acts as a vehicle, carrier or medium for the active ingredient. Thus, the compositions can be in the form of a tablet, pill, powder, lozenge, sachet, cachet, elixir, troche, aerosol (as a solid medium), soft and hard gelatin capsule, sterile packaged powder, dispensable powder, granule, or liquid.

Tablet forms can include, for example, one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents and pharmaceutically compatible carriers. In one embodiment of the present invention, the manufacturing processes may employ one or a combination of methods: (1) dry mixing, (2) direct compression, (3) milling, (4) dry or non-aqueous granulation, (5) wet granulation, or (6) fusion. Lachman et al., The Theory and Practice of Industrial Pharmacy (1986). Such tablets may also comprise film coatings, which disintegrate upon oral ingestion or upon contact with diluent.

In another embodiment of the present invention, solid compositions, such as tablets, are prepared by mixing an acid-labile pharmaceutical agent with a pharmaceutical excipient to form a solid preformulation composition containing a homogeneous mixture of the acid-labile pharmaceutical agent and a buffering agent of the present invention. When referring to these preformulation compounds as homogeneous, it is meant that the acid-labile pharmaceutical agent and buffering agent are dispersed evenly throughout the composition so that the composition may be readily subdivided into equally effective unit dosage forms, such as tablets, pills and capsules. This solid preformulation is then subdivided into unit dosage forms of the type described herein.

Compressed tablets are solid dosage forms prepared by compacting a formulation containing an acid-labile pharmaceutical agent and/or buffering agent and/or excipient selected to aid the processing and improve the properties of the product. The term “compressed tablet” generally refers to a plain, uncoated tablet for oral ingestion, prepared by a single compression or by pre-compaction tapping followed by a final compression.

The tablets or pills of the present invention may be coated or otherwise compounded to provide a dosage form affording the advantage of improved handling or storage characteristics. For example, the tablet or pill can comprise an inner dosage and an outer dosage component, the latter being in the form of an envelope over the former.

The term “suspension tablets” as used herein refers to compressed tablets which rapidly disintegrate after they are placed in water, and are readily dispersible to form a suspension containing a precise dose of the acid-labile pharmaceutical agent and/or buffering agent. Croscarmellose sodium is a known disintegrant for tablet formulations, and is available from FMC Corporation, Philadelphia, Pa. under the trademark Ac-Di-Sol®. It is frequently blended in compressed tableting formulations either alone or in combination with microcrystalline cellulose to achieve rapid disintegration of the tablet.

Illustratively, microcrystalline cellulose, alone or co-processed with other ingredients, is compressed into tablets and is known for its ability to improve compressibility of difficult to compress tablet materials. Commercially available products are available and can be used with the present invention. One example is available under the Avicel® trademark. Two different Avicel® products are utilized, Avicel® PH which is microcrystalline cellulose, and Avicel® AC-815, a co processed spray dried residue of microcrystalline cellulose and a calcium-sodium alginate complex in which the calcium to sodium ratio is in the range of about 0.40:1 to about 2.5:1. While AC-815 is comprised of 85% microcrystalline cellulose (MCC) and 15% of a calcium-sodium alginate complex, for purposes of the present invention this ratio may be varied from about 75% MCC to 25% alginate up to about 95% MCC to 5% alginate. Depending on the particular formulation and active ingredient, these two components may be present in approximately equal amounts or in unequal amounts, and either may comprise from about 10% to about 50% by weight of the tablet.

Dry oral formulations can contain such excipients as binders (for example, hydroxypropylmethylcellulose, polyvinyl pyrilodone, other cellulosic materials and starch), diluents (for example, lactose and other sugars, starch, dicalcium phosphate and cellulosic materials), disintegrating agents (for example, starch polymers and cellulosic materials) and lubricating agents (for example, stearates and talc).

Since the tablet may be used to form rapidly disintegrating chewable tablets, lozenges, troches or swallowable tablets; the intermediate formulations, as well as the process for preparing them, provide additional aspects of the present invention.

Effervescent tablets and powders are also prepared in accordance with the present invention. Effervescent salts have been used to disperse medicines in water for oral administration. Effervescent salts are granules or coarse powders containing a medicinal agent in a dry mixture, usually composed of sodium bicarbonate, citric acid and tartaric acid.

When the salts are added to water, the acids and the base react to liberate carbon dioxide gas, thereby causing “effervescence.”

The choice of ingredients for effervescent granules depends both upon the requirements of the manufacturing process and the necessity of making a preparation that disintegrates readily in water. Generally, the two needed ingredients are at least one acid and at least one base. The base releases carbon dioxide upon reaction with the acid. Examples of such acids include, but are not limited to, tartaric acid and citric acid. In one embodiment, the acid is a combination of both tartaric acid and citric acid. Examples of bases include, but are not limited to, sodium carbonate, potassium bicarbonate and sodium bicarbonate. In one embodiment, the base is sodium bicarbonate, and the effervescent combination has a pH of about 6.0 or higher.

Illustratively, effervescent salts include the following ingredients, which actually produce the effervescence: sodium bicarbonate, citric acid and tartaric acid. When added to water, the acids and base react to liberate carbon dioxide, resulting in effervescence. It should be noted that any acid-base combination that results in the liberation of carbon dioxide could be used in place of the combination of sodium bicarbonate and citric and tartaric acids, as long as the ingredients were suitable for pharmaceutical use and result in a pH of about 6.0 or higher.

It should be noted that it requires three molecules of NaHCO₃ to neutralize one molecule of citric acid and two molecules of NaHCO₃ to neutralize one molecule of tartaric acid. It is desired that the approximate ratio of ingredients is as follows:
Citric Acid:Tartaric Acid:Sodium Bicarbonate=1:2:3.44 (by weight). This ratio can be varied and continue to produce an effective release of carbon dioxide. For example, ratios of about 1:0:3 or 0:1:2 are also effective.

The method of preparation of the effervescent granules of the present invention employs three basic processes: wet granulation, dry granulation and fusion. The fusion method is used for the preparation of most commercial effervescent powders. It should be noted that, although these methods are intended for the preparation of granules, the formulations of effervescent salts of the present invention could also be prepared as tablets, according to known technology for tablet preparation.

Wet granulation is one the oldest method of granule preparation. The individual steps in the wet granulation process of tablet preparation include milling and sieving of the ingredients, dry powder mixing, wet massing, granulation and final grinding.

Dry granulation involves compressing a powder mixture into a rough tablet or “slug” on a heavy-duty rotary tablet press. The slugs are then broken up into granular particles by a grinding operation, usually by passage through an oscillation granulator. The individual steps include mixing of the powders, compressing (slugging) and grinding (slug reduction or granulation). No wet binder or moisture is involved in any of the steps.

Many other types of release delivery systems are available and known to those of ordinary skill in the art. They include polymer-based systems, such as polylactic and polyglycolic acid, polyanhydrides and polycaprolactone; nonpolymer systems that are lipids, including sterols, such as cholesterol, cholesterol esters and fatty acids, or neutral fats, such as mono-, di-and triglycerides; hydrogel release systems; silastic systems; peptide-based systems; wax coatings; compressed tablets using conventional binders (See, for example, Liberman et al., Pharmaceutical Dosage Forms, 2 Ed., Vol. 1, pp. 209-214 (1990), and excipients; partially fused implants; and the like. Specific examples include, but are not limited to: (a) erosional systems in which the polysaccharide is contained in a form within a matrix, found in U.S. Pat. No. 4,452,775; U.S. Pat. No. 4,667,014; and U.S. Pat. No. 4,748,034 and U.S. Pat. No. 5,239,660; and (b) diffusional systems in which an active component permeates at a controlled rate through a polymer, found in U.S. Pat. No. 3,832,253 and U.S. Pat. No. 3,854,480.

Liquid dosage forms for oral administration can include pharmaceutically acceptable emulsions, solutions, suspensions, syrups, and elixirs containing inert diluents commonly used in the art, such as water. Such compositions can also comprise, for example, wetting agents, emulsifying and suspending agents, and sweetening, flavoring, and perfuming agents. Also see, for example, U.S. Pat. No. 5,840,737, for liquid dosage forms.

Examples of suitable liquid dosage forms include, but are not limited, aqueous solutions comprising beta-cyclodextrin or a water soluble derivative of beta-cyclodextrin such as sulfobutyl ether beta-cyclodextrin; heptakis-2,6-di-O-methyl-beta-cyclodextrin; hydroxypropyl-beta-cyclodextrin; and dimethyl-beta-cyclodextrin.

In one embodiment of the present invention, the composition can further include an anti-foaming agent (for example, simethicone 80 mg, Mylicon®), and/or a parietal cell activator. Parietal cell activators such as chocolate, calcium and sodium bicarbonate and other alkaline substances, stimulate the parietal cells and enhance the pharmacologic activity of the proton pump inhibiting agent administered. For the purposes of this application, “parietal cell activator” or “activator” shall mean any compound or mixture of compounds possessing such stimulatory effect including, but not limited to, chocolate, sodium bicarbonate, calcium (for example, calcium carbonate, calcium gluconate, calcium hydroxide, calcium acetate and calcium glycerophosphate), peppermint oil, spearmint oil, coffee, tea and colas (even if decaffeinated), caffeine, theophylline, theobromine, and amino acids (particularly aromatic amino acids such as phenylalanine and tryptophan) and combinations thereof, and the salts thereof.

Such parietal cell activators are administered in an amount sufficient to produce the desired stimulatory effect without causing untoward side effects to patients. For example, chocolate, as raw cocoa, is administered in an amount of about 5 mg to 2.5 g per 20 mg dose of omeprazole (or equivalent pharmacologic dose of another proton pump inhibiting agent). The dose of activator administered to a subject, for example, a human, in the context of the present invention should be sufficient to effect a therapeutic response (that is, enhanced effect of proton pump inhibiting agent) over a desired time frame. The dose will be determined by the strength of the particular compositions employed and the condition of the person, as well as the body weight of the person to be treated. The size of the dose also will be determined by the existence, nature, and extent of any adverse side effects that might accompany the administration of a particular composition.

Illustratively, the approximate effective ranges for various parietal cell activators per 20 mg dose of omeprazole (or equivalent dose of other PPI) include, Chocolate (raw cocoa)—5 mg to 2.5 g; Sodium bicarbonate—7 mEq to 25 mEq; Calcium carbonate—1 mg to 1.5 g; Calcium gluconate—1 mg to 1.5 g; Calcium lactate—1 mg to 1.5 g; Calcium hydroxide—1 mg to 1.5 g; Calcium acetate—0.5 mg to 1.5 g; Calcium glycerophosphate—0.5 mg to 1.5 g; Peppermint oil—(powdered form) 1 mg to 1 g; Spearmint oil—(powdered form) 1 mg to 1 g; Coffee—20 ml to 240 ml; Tea—20 ml to 240 ml; Cola—20 ml to 240 ml; Caffeine—0.5 mg to 1.5 g; Theophylline—0.5 mg to 1.5 g; Theobromine—0.5 mg to 1.5 g; Phenylalanine—0.5 mg to 1.5 g; and Tryptophan—0.5 mg to 1.5 g.

In one embodiment of the present invention, the composition is administered to a subject in a gastrointestinal-disorder-effective amount, that is, the composition is administered in an amount that achieves a therapeutically-effective dose of a proton pump inhibiting agent in the blood serum of a subject for a period of time to elicit a desired therapeutic effect. Illustratively, in a fasting adult human (fasting for generally at least 10 hours) the composition is administered to achieve a therapeutically-effective dose of a proton pump inhibiting agent in the blood serum of a subject from about 5 minutes after administration of the composition. In another embodiment of the present invention, a therapeutically-effective dose of the proton pump inhibiting agent is achieved in the blood serum of a subject at about 10 minutes from the time of administration of the composition to the subject. In another embodiment of the present invention, a therapeutically-effective dose of the proton pump inhibiting agent is achieved in the blood serum of a subject at about 20 minutes from the time of administration of the composition to the subject. In yet another embodiment of the present invention, a therapeutically-effective dose of the proton pump inhibiting agent is achieved in the blood serum of a subject at about 30 minutes from the time of administration of the composition to the subject. In still another embodiment of the present invention, a therapeutically-effective dose of the proton pump inhibiting agent is achieved in the blood serum of a subject at about 40 minutes from the time of administration of the composition to the subject.

In one embodiment of the present invention, a therapeutically-effective dose of the proton pump inhibiting agent is achieved in the blood serum of a subject at about 10 minutes to about 12 hours from the time of administration of the composition to the subject. In another embodiment of the present invention, a therapeutically-effective dose of the proton pump inhibiting agent is achieved in the blood serum of a subject at about 20 minutes to about 6 hours from the time of administration of the composition to the subject. In yet another embodiment of the present invention, a therapeutically-effective dose of the proton pump inhibiting agent is achieved in the blood serum of a subject at about 20 minutes to about 2 hours from the time of administration of the composition to the subject. In still another embodiment of the present invention, a therapeutically-effective dose of the proton pump inhibiting agent is achieved in the blood serum of a subject at about 40 minutes to about 2 hours from the time of administration of the composition to the subject. And in yet another embodiment of the present invention, a therapeutically-effective dose of the proton pump inhibiting agent is achieved in the blood serum of a subject at about 40 minutes to about 1 hour from the time of administration of the composition to the subject.

Contemplated compositions of the present invention provide a therapeutic effect as proton pump inhibiting agent medications over an interval of about 5 minutes to about 24 hours after administration, enabling once-a-day or twice-a-day administration if desired. The amount of therapeutic agent necessary to elicit a therapeutic effect can be experimentally determined based on, for example, the absorption rate of the agent into the blood serum, the bioavailability of the agent, and the amount of protein binding of the agent. It is understood, however, that specific dose levels of the therapeutic agents of the present invention for any particular subject depends upon a variety of factors including the activity of the specific compound employed, the age, body weight, general health, sex, and diet of the subject (including, for example, whether the subject is in a fasting or fed state), the time of administration, the rate of excretion, the drug combination, and the severity of the particular disorder being treated and form of administration. Treatment dosages generally may be titrated to optimize safety and efficacy. Typically, dosage-effect relationships from in vitro and/or in vivo tests initially can provide useful guidance on the proper doses for subject administration. Studies in animal models generally may be used for guidance regarding effective dosages for treatment of gastrointestinal disorders or diseases in accordance with the present invention. In terms of treatment protocols, it should be appreciated that the dosage to be administered will depend on several factors, including the particular agent that is administered, the route administered, the condition of the particular subject, etc. Generally speaking, one will desire to administer an amount of the compound that is effective to achieve a serum level commensurate with the concentrations found to be effective in vitro for a period of time effective to elicit a therapeutic effect. Thus, where a compound is found to demonstrate in vitro activity at, for example, 10 ng/ml, one will desire to administer an amount of the drug that is effective to provide at least about a 10 ng/ml concentration in vivo for a period of time that elicits a desired therapeutic effect, for example, raising of gastric pH, reducing gastrointestinal bleeding, reducing the need for blood transfuision, improving survival rate, more rapid recovery, parietal cell activation and H⁺, K⁺-ATPase inhibition or improvement or elimination of symptoms, and other indicators as are selected as appropriate measures by those skilled in the art. Determination of these parameters is well within the skill of the art. These considerations are well known in the art and are described in standard textbooks.

In order to measure and determine the gastrointestinal disorder-or disease-effective amount of a proton pump inhibiting agent to be delivered to a subject, serum proton pump inhibiting agent concentrations can be measured using standard assay techniques, such as, for example, HPLC.

In one embodiment of the present invention, the controlled-release component comprises a substituted benzimidazole in the form of a granule or core. Enteric coatings suitable for application directly to the granule or core are generally an enteric coating that is a gastric acid resistant polymer such as cellulose acetate phthalate, hydroxypropylmethylcellulose acetate succinate, hydroxypropylmethylcellulose phthalate, polyvinylacetate phthalate, carboxymethylethylcellulose, acrylic acid polymers and copolymers, methacrylic acid polymers and copolymers. Furthermore, dosage forms wherein the enteric coating is applied directly (that is, in the absence of a subcoating) to the granule or core is within the scope of the present invention.

In one embodiment of the present invention, the enteric coating having a defined composition and/or a thickness, is applied to a portion of the composition of the present invention in which it is effective to render that portion of the composition impermeable to gastrointestinal fluid until a predetermined pH is reached. In one embodiment, the controlled-component remains impermeable to gastrointestinal fluid having the above respective pH's for a period of time of about 30 to 45 seconds, or about 1, 2, 3, 4, 5, 6, 7, 8, 9, 10, 11, 12, 13, 14, 15, 16, 17, 18, 19, 20, 30, 40, 50, 60, 70, 80, or 90 minutes after exposure to the fluid. The particular desired pH is generally compound specific and depends upon, among other things, its particular pKa and other chemical properties.

In some embodiments, the compositions of the present invention have improved bioavailability relative to current formulations known in the art. In one embodiment, the present invention relates to an omeprazole dosage form which has improved bioavailability relative to the omeprazole formulation which is the subject of U.S. Food and Drug Administration approved New Drug Application 19810, and to a lansoprazole dosage form which has improved bioavailability relative to the lansoprazole formulation which is the subject of U.S. Food and Drug Administration approved New Drug Application 20406. It is believed, although not certain, and without being bound to any particular theory, that the present compositions have improved bioavailability relative to the commercial formulations which contain enteric coated granules or pellets because a portion of the granules or pellets in the commercial products release their contents in the stomach and the active ingredient is decomposed before it is absorbed into the bloodstream.

In one embodiment of the present invention, the buffering agent and the controlled release component are dry blended and compressed-into a mass composition, such as a tablet or granule, having a hardness sufficient to cause the composition to disintegrate within 30 seconds after exposure to gastrointestinal fluid, for example, after oral administration to a subject or testing the composition in an in vitro stomach model, thereby releasing the buffering agent and the controlled-release component into the gastrointestinal fluid, which disintegrates within.

The present invention also is directed to a therapeutic method of treating a condition or disorder where treatment with an inhibitor of H⁺, K⁺-ATPase, such as a proton pump inhibiting agent, is indicated. The method comprises the oral administration of one or more of the pharmaceutical compositions of the present invention to a subject in need thereof. Treatment is generally continued as necessary over a period of hours, days, weeks to several months or years until the condition or disorder has been controlled or eliminated. Subjects undergoing treatment with the compositions disclosed herein can be routinely monitored by any of the methods well known in the art to determine the effectiveness of therapy. Continuous analysis of such data permits modification of the treatment regimen during therapy so that optimal effective amounts of compounds of the present invention are administered at any point in time, and so that the duration of treatment can be determined as well. In this way, the treatment regimen/dosing schedule can be rationally modified over the course of therapy so that the lowest amount of an inhibitor of H⁺, K⁺-ATPase exhibiting satisfactory effectiveness is administered, and so that administration is continued only so long as is necessary to successfully treat the condition or disorder.

In one embodiment of the present invention, the compositions are designed to produce release of an acid-labile pharmaceutical agent to the site of delivery (typically the stomach), while preventing acid degradation of the acid-labile pharmaceutical agent. Acid-labile pharmaceutical agents, for example, can be formulated or coadministered with one or more buffering agents sufficient to protect the acid-labile pharmaceutical agent in an environment with the ultimate goal being to deliver an acid-labile pharmaceutical agent to the stomach (or other environment) either via a liquid, a powder or solid dosage form that produces an immediate-release of buffering agent from the composition before, during or after the release of the acid-labile pharmaceutical agent, such that the acid-labile pharmaceutical agent is quickly available for absorption in a substantially non-acid degraded or reacted form. Accordingly, Applicant has found that certain amounts of buffering agents co-administered or mixed with certain acid-labile pharmaceutical agents prevent acid degradation of the acid-labile pharmaceutical agent when the buffering agent produces a pH in the gastrointestinal fluid, for example, the stomach or other site of administration, that is equal to the pKa of the acid-labile pharmaceutical agent plus an amount sufficient to provide undegraded and bioactive acid-labile pharmaceutical agent to the blood upon administration (for example, an increase of about a 0.7 log value will reduce the degradation to about 10%). The pKa is defined as the pH at which about 50% of a chemical is in the ionized form. When the pH of the environment equals the pKa of the acid-labile pharmaceutical agent, then 50% ionization (degradation) of the acid-labile pharmaceutical agent occurs. However, by adding the factor of about 0.7, this ionization is reduced to about 90%. Such buffering agents should interact with hydrogen ion at rates that exceed the interaction with the acid-labile pharmaceutical agent. Thus, the solubility of the buffering agents and acid-labile pharmaceutical agents are considerations because solubility often determines the rate of interaction of H+ ion with another compound.

In one embodiment of the present invention, a buffering agent includes a buffering agent or combination of buffering agents that interacts with hydrochloric acid (or other acids in the environment of interest) faster than the acid-labile pharmaceutical agent interacts with the same acids. When placed in a liquid phase (usually in water), the buffering agent produces and maintains a pH greater than the pKa of the acid-labile pharmaceutical agent. In one embodiment, by raising the pH of the environment to the same of the pKa of the acid-labile pharmaceutical agent plus about 0.7 log value (or greater), the expected degradation (ionization) can be reduced from about 50% to about 10%. A log value of about 0.7 is added to the lowest pH of the environment of interest needed to minimize or eliminate the acid-induced degradation of the acid-labile pharmaceutical agent, which represents a decrease of about 5.01187% in stability of the acid-labile pharmaceutical agent from 1 log value, thus resulting in a stability of approximately 90%, a value widely accepted as desirable in pharmaceutical products. In many cases it is permissible to accept a value less than 0.7, as long as a therapeutically-effective amount of the acid-labile pharmaceutical agent is absorbed into the bloodstream of a subject.

As mentioned above, the pKa of a given acid-labile pharmaceutical agent indicates inherent stability with respect to acid degradation; the lower the pKa, the more stable the acid-labile pharmaceutical agent. The solubility of the acid-labile pharmaceutical agent will also dictate the rate at which the acid-labile pharmaceutical agent complexes with, and is degraded by, acid. These two physicochemical characteristics (pKa and solubility) of the acid-labile pharmaceutical agent interact with the physicochemical characteristics of the buffering agents (pH, buffering capacity and rate of buffering action) in the presence of acid in the environment to determine the degradation of the acid-labile pharmaceutical agent over time. The less soluble an acid-labile pharmaceutical agent is in water, in general the lower the initial degradation when placed in an acidic environment. The following Table No. 15 elaborates on the time for 50% of drug to be degraded (t ½), pKa and solubility in water of several proton pump inhibiting agents.

TABLE NO. 15
Acid Degradation over Time

| | Pantoprazole | | | Rabeprazole |
|---|---|---|---|---|
| pH | sodium | Omeprazole | Lansoprazole | sodium |
| 1.2 | 4.6 min | 2.8 min | 2.0 min | 1.3 min |
| 5 | 2.8 hr | 1.0 hr | 1.1 hr | |
| 5.1 | 4.7 hr | 1.4 hr | 1.5 hr | 7.2 minutes |
| 6 | 21 hr | 7.3 hr | 6.4 hr | |
| 7 | 73 hr | 39 hr | 35 hr | |
| pKa | 3 | 3.9 | 4.1 | 4.9 |
| Solubility | very soluble | slightly | very slightly | Very soluble |
| | | soluble | soluble | |

See, Kromer W, et al. Differences in pH-Dependent Activation Rates of Substituted Benzimidazoles and Biological in vitro Correlates, PHARMACOLOGY 1998; 56:57-70.

Although not wishing to be bound by theory, pantoprazole sodium, with a pKa of 3, is believed to be inherently more stable in an acidic environment than the other proton pump inhibiting agents, as it is also very soluble in water and thus could undergo 50% degradation in an acidic stomach with a pH of 1.2 in less than 5 minutes. Therefore, in one embodiment of the present invention, the buffering agent(s) used with pantoprazole sodium, interact with H+ion (or other acidic substances) more rapidly than the pantoprazole sodium interacts with such acids and maintain the rapid complexation through the dwell time. In yet another embodiment, the overall pH of the gastric contents is kept at least at the pKa+0.7 (that is, pH 3.7) from the time the proton pump inhibiting agent in solution comes into contact with the gastric acid continuing throughout the dwell time. In one embodiment, buffering agents for formulations of pantoprazole sodium include those buffering agents whose conjugate acids possess a pKa>3.7 and which are very soluble (for example, potassium bicarbonate and sodium bicarbonate). Another formulation method for pantoprazole is to decrease its solubility such as by selecting a less soluble salt form or the non-salt form, pantoprazole.

Rabeprazole sodium is also very soluble in water and could undergo 50% degradation in an acidic stomach with a pH of 1.2 in less than 1.5 minutes. It is not very stable to acid degradation due to its higher pKa of 4.9. In one embodiment of the present invention, a buffering agent(s) used with rabeprazole sodium interacts with H+ ion (or other acidic substances) more rapidly than the rabeprazole sodium interacts with such acids to prevent early degradation, and possesses high neutralizing capacity to enable rabeprazole to survive through. the dwell time. Illustratively, sodium or potassium bicarbonate would be good choices in this instance.

Another option for rabeprazole sodium (as well as any sodium salt of a proton pump inhibitor, which would tend to be more soluble than the base form) is to reduce the solubility of rabeprazole sodium when in aqueous form such as using a less soluble salt form or using the non-salt form. This decreases early degradation because the rabeprazole must first undergo dissolution in water before it is degraded by acid. In this embodiment, the suitable buffering agent for rabeprazole sodium should possess high neutralizing capacity to enable rabeprazole to survive through the dwell time.

The dosage form may affect the suitability of a buffering agent for use in a formulation. For example, magnesium oxide is a buffering agent with high buffering capacity but slow onset when formulated as a tablet. However, when formulated as a powder, or a tablet of low compression, or with tablet disintegrants such as pregelatinized starch, it disintegrates more rapidly.

Omeprazole base is only slightly soluble in water and, as such, less of the drug is subject to early and continued degradation. The soluble portion of omeprazole is vulnerable to early degradation in the gastric environment. Dissolution of the remaining insoluble portion is expected to occur within minutes of encountering the water of the gastric secretions. This dissolution time provides some protection against early degradation provided that relatively low volumes of water are used during delivery or in the product formulation. After several minutes in the gastric environment, upon complete dissolution, omeprazole could undergo 50% degradation in less than 3 minutes. Omeprazole is moderately stable owing to its pKa of 3.9. Illustratively, a suitable buffering agent for omeprazole is rapid acting and possesses at least moderate neutralizing capacity to enable omeprazole to survive through the dwell time.

Lansoprazole base is very slightly soluble in water and, as such, less of the drug is subject to early degradation. The soluble portion is vulnerable to early degradation. Dissolution of the remaining insoluble portion is expected to occur within several minutes of encountering the water of the gastric secretions. This dissolution time provides some protection against early degradation provided that relatively low volumes of water are used for delivery or in the product formulation. After several minutes, upon complete dissolution, lansoprazole could undergo 50% degradation in 2 minutes. Lansoprazole is moderately stable owing to its pKa of 4.1. Illustratively, a suitable buffering agent for lansoprazole is rapid acting, and possesses moderate to high neutralizing capacity to enable lansoprazole to survive through the dwell time. In one embodiment, the pH of the gastrointestinal fluid is kept at greater than about 4.8 from the time the proton pump inhibiting agent in solution comes into contact with the gastric acid continuing throughout the dwell time.

As used herein, “rapid acting” in the context of a buffering agent means a buffering agent that raises the pH of the environment to greater than or equal to the sum of the pKa of an acid-labile pharmaceutical agent plus about 0.7 in a time sufficient to prevent significant degradation of the acid-labile pharmaceutical agent. In one embodiment, the rapid acting buffering agent raises the pH to at least the pKa of the proton pump inhibiting agent plus 0.7 log value within 10 minutes.

The present methods, kits, and compositions can also be used in combination (“combination therapy”) with another pharmaceutical agent that is indicated for treating or preventing a gastrointestinal disorder, such as, for example, an anti-bacterial agent, an irritable bowel syndrome drug, a motility agent, an anti-emetic agent, an alginate, a prokinetic agent, a H₂-antagonist, an antacid, or sucralfate, which are commonly administered to minimize the pain and/or complications related to this disorder. Illustratively, such drugs include metoclopramide, Lotrenex®, mesalamine (5-ASA), prednisone, ranitidine and cimetidine. . These drugs have certain disadvantages associated with their use. Some of these drugs are not completely effective in the treatment of the aforementioned conditions and/or produce adverse side effects, such as mental confusion, constipation, diarrhea, and thrombocytopenia. H₂-antagonists, such as ranitidine and cimetidine, are relatively costly modes of therapy, particularly in NPO patients, which frequently require the use of automated infusion pumps for continuous intravenous infusion of the drug. However, when used in conjunction with the present invention, that is, in combination therapy, many if not all of these unwanted side effects can be reduced or eliminated. The reduced side effect profile of these drugs is generally attributed to, for example, the reduce dosage necessary to achieve a therapeutic effect with the administered combination.

The phrase “combination therapy” embraces the administration of a composition of the present invention in conjunction with another pharmaceutical agent that is indicated for treating or preventing a gastrointestinal disorder in a subject, as part of a specific treatment regimen intended to provide a beneficial effect from the co-action of these therapeutic agents for the treatment of a gastrointestinal disorder. The beneficial effect of the combination includes, but is not limited to, pharnacokinetic or pharmacodynamic co-action resulting from the combination of therapeutic agents. Administration of these therapeutic agents in combination typically is carried out over a defined time period (usually substantially simultaneously, minutes, hours, days, weeks, months or years depending upon the combination selected). “Combination therapy” generally is not intended to encompass the administration of two or more of these therapeutic agents as part of separate monotherapy regimens that incidentally and arbitrarily result in the combinations of the present invention. “Combination therapy” is intended to embrace administration of these therapeutic agents in a sequential manner, that is, where each therapeutic agent is administered at a different time, as well as administration of these therapeutic agents, or at least two of the therapeutic agents, in a substantially simultaneous manner. Substantially simultaneous administration can be accomplished, for example, by administering to the subject a single tablet or capsule having a fixed ratio of each therapeutic agent or in multiple, single capsules, or tablets for each of the therapeutic agents. Sequential or substantially simultaneous administration of each therapeutic agent can be effected by any appropriate route. The composition of the present invention can be administered orally or nasogastric, while the other therapeutic agent of the combination can be administered by any appropriate route for that particular agent, including, but not limited to, an oral route, a percutaneous route, an intravenous route, an intramuscular route, or by direct absorption through mucous membrane tissues. For example, the composition of the present invention is administered orally or nasogastric and the therapeutic agent of the combination may be administered orally, or percutaneously. The sequence in which the therapeutic agents are administered is not narrowly critical. “Combination therapy” also can embrace the administration of the therapeutic agents as described above in further combination with other biologically active ingredients, such as, but not limited to, a pain reliever, such as a steroidal, such as an opiate or opiod, or nonsteroidal anti-inflammatory drug, or an agent for improving stomach motility, for example, and with non-drug therapies, such as, but not limited to, surgery.

Illustratively, an antacid of interest that can be used in the methods, kits, combinations, and compositions of the present invention includes, but is not limited to, alexitol sodium, almagate, aluminum hydroxide, aluminum magnesium silicate, aluminum phosphate, azulene, basic aluminum carbonate gel, bismuth aluminate, bismuth phosphate, bismuth subgallate, bismuth subnitrate, dihydroxyaluminum aminoacetate, dihydroxyaluminum sodium carbonate, ebimar, magaldrate, magnesium carbonate hydroxide, magnesium oxide, magnesium peroxide, magnesium phosphate tribasic, magnesium silicate, potassium citrate, and combinations thereof. (Based in part upon the list provided in The Merck Index, Merck & Co. Rahway, N.J. (2001)).

The therapeutic compounds which make up the combination therapy may be a combined dosage form or in separate dosage forms intended for substantially simultaneous administration. The therapeutic compounds that make up the combination therapy may also be administered sequentially, with either therapeutic compound being administered by a regimen calling for two step administration. Thus, a regimen may call for sequential administration of the therapeutic compounds with spaced-apart administration of the separate, active agents. The time period between the multiple administration steps may range from, for example, a few minutes to several hours to days, depending upon the properties of each therapeutic compound such as potency, solubility, bioavailability, plasma half-life and kinetic profile of the therapeutic compound, as well as depending upon the effect of food ingestion and the age and condition of the subject. Circadian variation of the target molecule concentration may also determine the optimal dose interval. The therapeutic compounds of the combined therapy whether administered simultaneously, substantially simultaneously, or sequentially, may involve a regimen calling for administration of one therapeutic compound by oral route and another therapeutic compound by an oral route, a percutaneous route, an intravenous route, an intramuscular route, or by direct absorption through mucous membrane tissues, for example. Whether the therapeutic compounds of the combined therapy are administered orally, by inhalation spray, rectally, topically, buccally (for example, sublingual), or parenterally (for example, subcutaneous, intramuscular, intravenous and intradermal injections, or infusion techniques), separately or together, each such therapeutic compound will be contained in a suitable pharmaceutical formulation of pharmaceutically-acceptable excipients, diluents or other formulations components.

The present invention is further illustrated by the following examples, which should not be construed as limiting in any way. The experimental procedure to generate the data shown are discussed in more detail below. The symbols and conventions used in these examples are consistent with those used in the contemporary pharmaceutical literature. Unless otherwise stated, (i) all percentages recited in these examples are weight percents based on total composition weight, and (ii) total composition weight for capsules is the total capsule fill weight and does not include the weight of the actual capsule employed.

### EXAMPLES

For all formulations herein, multiple doses may be proportionally compounded as is known in the art.

### Example 1

### pH Dependent Controlled Release Composition

The release profile of a composition containing a pH-dependent controlled-release component containing an enteric coating is determined according to the following procedure: Dissolution testing is conducted with a USP Apparatus II (paddles at 50 rpm) using a one-stage dissolution medium of 50 ml 0.1 N hydrochloric acid at 37° C. Drug release with time is determined by HPLC on samples pulled at selected intervals.

In one embodiment of the present invention, microgranules of omeprazole are coated with Eudragit L30 D-55. The enterically coated microgranules are then combined with one or more suitable buffering agents and optionally one or more suitable excipients.

Upon consumption, the antacid present in the pharmaceutical composition is released in the stomach, which raises the pH of the gastrointestinal fluid and allows for the disintegration of the enteric coat. Once the enteric coat disintegrates, the acid labile pharmaceutical agent is released. In some examples, the enteric coating acts as a controlled-release layer and provides a lag time of release of the acid-labile pharmaceutical agent of about 30 seconds to about 60 minutes. Illustratively, a release profile for a controlled-release formulation of the present invention when tested by the one-stage in vitro dissolution medium described above are provided below in Table Nos. 16 and 17.

TABLE NO. 16
Release Profile of Controlled Release Formulation-
Sodium Bicarbonate (15 mEq)/
Calcium Carbonate (15 mEq) Buffer

| | pH of Gastrointestinal | % Acid-labile Pharmaceutical |
|---|---|---|
| Time | Fluid | Agent Release |
| 1 minute | 2.0 | — |
| 5 minutes | >5.5 | 2% |
| 10 minutes | >5.5 | 5% |
| 15 minutes | >5.5 | 10% |
| 20 minutes | >5.5 | 15% |
| 25 minutes | >5.5 | 30% |
| 30 minutes | >5.5 | 55% |
| 35 minutes | >5.5 | 80% |

TABLE NO. 17
Release Profile of Controlled Release Formulation-
Sodium Bicarbonate (20 mEq) Buffer

| | pH of Gastrointestinal | % Acid-labile Pharmaceutical |
|---|---|---|
| Time | Fluid | Agent Release |
| 1 minute | 4.0 | — |
| 5 minutes | >5.5 | 2% |
| 10 minutes | >5.5 | 5% |
| 15 minutes | >5.5 | 10% |
| 20 minutes | >5.5 | 15% |
| 25 minutes | >5.5 | 30% |
| 30 minutes | >5.5 | 55% |
| 35 minutes | >5.5 | 80% |

The invention has been described in an illustrative manner, and it is to be understood that the terminology used is intended to be in the nature of description rather than of limitation. All patents and other references cited herein are incorporated herein by reference in their entirety. Many modifications, equivalents, and variations of the present invention are possible in light of the above teachings, therefore, it is to be understood that within the scope of the appended claims, the invention may be practiced other than as specifically described.

1. A solid pharmaceutical composition, comprising:
(a) a gastrointestinal-disorder-effective amount of at least one enteric-coated proton pump inhibitor which proton pump inhibitor is acid labile; and
(b) at least one buffering agent; wherein upon oral administration, the enteric coating substantially dissolves in gastrointestinal fluid; and
the amount of buffering agent is sufficient to protect at least a therapeutically effective amount of the proton pump inhibitor from acid degradation in the gastrointestinal fluid.

2. The composition of claim 1, wherein the proton pump inhibitor is selected from omeprazole, tenatoprazole, lansoprazole, rabeprazole, esomeprazole, pantoprazole, pariprazole, and leminoprazole, and a salt, an ester, a hydrate, an amide, an enantiomer, an isomer, a tautomer, a prodrug, a polymorph, or a derivative thereof.

3. The composition of claim 1, wherein the proton pump inhibitor is selected from omeprazole, lansoprazole, esomeprazole, and a salt, an ester, a hydrate, an amide, an enantiomer, an isomer, a tautomer, a prodrug, a polymorph, or a derivative thereof.

4. The composition of claim 1, wherein the proton pump inhibitor is in an amount of about 2 mg to about 300 mg.

5. The composition of claim 2, wherein the proton pump inhibitor is in an amount selected from 5 mg, 10 mg, 15 mg, 20 mg, 30 mg, 40 mg, and 60 mg.

6. The composition of claim 1, wherein the proton pump inhibitor is micronized.

7. The composition of claim 1, wherein the composition is administered in an amount to achieve an initial serum concentration of the proton pump inhibitor greater than about 0.1 μg/ml at any time within about 1 hour after administration of the composition.

8. The composition of claim 1, wherein the composition is administered in an amount to maintain a serum concentration of the proton pump inhibitor greater than about 0.1 μg/ml from about 30 minutes after oral administration of the composition to a subject.

9. The composition of claim 1, wherein upon oral administration to a subject, the composition provides a pharmacokinetic profile such that at least about 50% of total area under serum concentration time curve (AUC) for the proton pump inhibitor occurs within about 1.5 hours after administration of a single dose of the composition to the subject.

10. The composition of claim 1, wherein upon oral administration to a subject, the composition provides a pharmacokinetic profile such that the proton pump inhibitor reaches a maximum serum concentration within about 1 hour after administration of a single dose of the composition.

11. The composition of claim 1, wherein the buffering agent is present in an amount to adjust the pH of the gastrointestinal fluid to between about 3 to about 8 after ingestion by a subject.

12. The composition of claim 1, wherein the at least one buffering agent is selected from aluminum hydroxide, aluminum hydroxide/magnesium carbonate, aluminum hydroxide/magnesium carbonate/calcium carbonate co-precipitate, aluminum magnesium hydroxide, aluminum hydroxide/magnesium hydroxide co-precipitate, aluminum hydroxide/sodium bicarbonate coprecipitate, calcium acetate, calcium bicarbonate, calcium borate, calcium carbonate, calcium citrate, calcium chloride, calcium glycerophosphate, calcium hydroxide, calcium lactate, calcium phthalate, calcium phosphate, calcium succinate, calcium tartrate, dibasic sodium phosphate, dipotassium hydrogen phosphate, dipotassium phosphate, disodium hydrogen phosphate, disodium succinate, dry aluminum hydroxide gel, magnesium acetate, magnesium aluminate, magnesium borate, magnesium bicarbonate, magnesium carbonate, magnesium citrate, magnesium hydroxide, magnesium lactate, magnesium metasilicate aluminate, magnesium oxide, magnesium phthalate, magnesium phosphate, magnesium silicate, magnesium succinate, magnesium tartrate, potassium acetate, potassium carbonate, potassium bicarbonate, potassium borate, potassium citrate, potassium metaphosphate, potassium phthalate, potassium phosphate, potassium polyphosphate, potassium pyrophosphate, potassium succinate, potassium tartrate, sodium acetate, sodium bicarbonate, sodium borate, sodium carbonate, sodium citrate, sodium dihydrogen phosphate, sodium hydrogen phosphate, sodium hydroxide, sodium lactate, sodium phthalate, sodium phosphate, sodium polyphosphate, sodium pyrophosphate, sodium sesquicarbonate, sodium succinate, sodium tartrate, sodium tripolyphosphate, synthetic hydrotalcite, tetrapotassium pyrophosphate, tetrasodium pyrophosphate, trihydroxymethylaminomethane, tripotassium phosphate, trisodium phosphate, and trometamol; and combinations thereof.

13. The composition of claim 1, wherein the at least one buffering agent is selected from sodium bicarbonate, sodium carbonate, calcium carbonate, magnesium oxide, magnesium hydroxide, magnesium carbonate, aluminum hydroxide, and mixtures thereof.

14. The composition of claim 1, wherein the at least one buffering agent is in an amount of at least about 2 mEq.

15. The composition of claim 1, wherein the at least one buffering agent is in an amount of about 2 mEq to about 40 mEq.

16. The composition of claim 1, wherein the at least one buffering agent is in an amount from about 250 mg to about 3000 mg.

17. The composition of claim 1, wherein the enteric coating comprises at least one of acetylatedmonoglyceride, carboxymethyl cellulose, cellulose acetate, cellulose acetate butyrate, cellulose acetate phthalate, cellulose acetate trimellitate, cetyl alcohol, citric acid anhydrous, diethyl phthalate, diglyceride, ethyl cellulose, Eudragit® L-30D-55, Eudragit® NE30D, Eudragit® L 100, Eudragit® L 100-55, Eudragit® S100, Eudragit® FS 30 D, glyceryl monostearate, hydrogen peroxide, hydroxypropyl cellulose, hydroxypropyl methylcellulose, hydroxypropyl methylcellulose phthalate, hydroxypropyl methylcellulose acetate succinate, KollICoat® MAE30DP, Macrogel® 6000, methacrylic acid copolymer, monoglyceride, an organic acid, polyethylene glycol, polyethylene glycol 400, polyethylene glycol 6000, polymethacrylates containing carboxyl groups, polymethacrylates containing quaternary ammonium groups, polyquid PA-30, polysobate 80, polyvinyl acetate phthalate, polyvinyl alcohol, polyvinylpyrrolidone, a salt, shellac, sodium laurylsulphate, stearyl alcohol, a sugar, talc, triacetin, triethyl citrate, Tween® 80, a wax, or zein.

18. The composition of claim 1, wherein the enteric coating has a thickness of about 0.001 microns to about 100 microns.

19. The composition of claim 1, wherein the enteric coating has a thickness of about 0.01 microns to about 50 microns.

20. The composition of claim 1, wherein the enteric coating has a thickness less than about 25 microns.

21. The composition of claim 1, wherein the enteric coating has a thickness less than about 10 microns.

22. The composition of claim 1, wherein the enteric coating is a thickness that provides for release of at least 80% of the proton pump inhibitor in vitro within about 120 minutes.

23. The composition of claim 1, wherein the enteric coating is a thickness that provides for release of at least 80% of the proton pump inhibitor in vitro within about 60 minutes.

24. The composition of claim 1, wherein the enteric coating is a thickness that provides for release of at least 50% of the proton pump inhibitor in vitro within about 120 minutes

25. The composition of claim 1, wherein the enteric coating is a thickness that provides for release of at least 50% of the proton pump inhibitor in vitro within about 60 minutes.

26. The composition of claim 1, wherein the composition is in a pharmaceutical dosage form selected from a tablet, chewable tablet, caplet, a powder, a pill, a capsule, a lozenge, a sachet, a cachet, a troche, a minitab in a capsule, a pellet, and a granule.

27. The composition of claim 1, wherein the composition further comprises at least one of an excipient, a pharmaceutically compatible carrier, a binder, a filling agent, suspending agent, a taste masking agent, a flavoring agent, a sweetening agent, a disintegrant, a flow aid, a lubricant, an adjuvant, a colorant, a diluent, a solubilizer, a moistening agent, a stabilizer, a wetting agent, an anti-adherent, a glidant, a preservative, a parietal cell activator, an anti-foaming agent, an antioxidant, a chelating agent, an antifungal agent, an antibacterial agent, or an isotonic agent.

28. A method of treating a condition or disorder where treatment with a proton pump inhibitor is indicated, the method comprising orally administering the composition according to claim 1 to a subject in need of such treatment.

29. The method of claim 28, wherein the gastrointestinal disorder is a duodenal ulcer disease, a gastric ulcer disease, a gastroesophageal reflux disease, an erosive esophagitis, a poorly responsive symptomatic gastroesophageal reflux disease, a pathological gastrointestinal hypersecretory disease, Zollinger Ellison Syndrome, acid dyspepsia, heartburn, an esophageal disorder, a non-erosive reflux disorder, or an NSAID induced ulcer.
